(19) United States
(12) Patent Application Publication
(10) Pub. No.: US 2026/0034354 A1
(43) Pub. Date: Feb. 5, 2026

(54) MEDICAL DEVICES CONFIGURED FOR THERAPEUTIC ELECTROPORATION OF BIOLOGIC TISSUES

(71) Applicant: Autonomix Medical, Inc., Ivyland, PA (US)

(72) Inventors: Landy Toth, Doylestown, PA (US); Robert S. Schwartz, Inver Grove Heights, MN (US)

(21) Appl. No.: 19/099,897

(22) PCT Filed: Aug. 1, 2023
(86) PCT No.: PCT/US2023/029193

§ 371 Date: Jan. 30, 2025

Related U.S. Application Data
(60) Provisional application No. 63/394,705, filed on Aug. 3, 2022.

(51) Int. Cl. A61N 1/32; A61M 5/142; A61M 25/10; A61N 1/05

(52) U.S. Cl. CPC A61N 1/327; A61M 5/142; A61M 25/1011; A61M 25/10184; A61N 1/0502; A61M 2025/1052; A61M 2205/3327; A61M 2210/12

(57) ABSTRACT

A medical device comprises an elongate member, one or more expandable elements positioned on the elongate member, one or more ports, one or more electrodes, and a controller. The controller is configured to actuate the one or more expandable elements within a vessel of a subject to block fluid flow through the vessel downstream to a target tissue site, to deliver a bolus of conducting medium via the one or more ports towards the target tissue site, and to apply energy pulses via the one or more electrodes for therapeutic electroporation while the bolus of the conducting medium is in contact with the one or more electrodes and extends from the one or more electrodes towards the target tissue site.

### BACKGROUND

### Technical Field

The present disclosure relates to medical devices, such as delivery catheters, and to systems and methods for performing therapeutic electroporation of biologic tissues using such medical devices.

### Background

Various approaches have been developed to deliver energy to target tissues within a subject, where the energy may be delivered for neuromodulation, denervation and/or ablation of the target tissues. Some approaches are catheter-based, and involve a deployable mechanical structure with hard, metallic electrodes that need to conform to the surface of the target tissues to be treated. As vessels narrow at the parenchyma of an organ, near to a tumor site, etc., it is challenging to reduce the size of the mechanical components and electrodes of a catheter-based medical device to reach the target tissue while also being able to maintain an effective treatment volume.

### SUMMARY

Illustrative embodiments provide methods and medical devices for therapeutic electroporation of biologic tissues.

In some embodiments, a medical device comprises an elongate member, one or more expandable elements positioned on the elongate member, one or more ports, one or more electrodes, and a controller. The controller is configured to actuate the one or more expandable elements within a vessel of a subject to block fluid flow through the vessel downstream to a target tissue site, to deliver a bolus of conducting medium via the one or more ports towards the target tissue site, and to apply energy pulses via the one or more electrodes for therapeutic electroporation while the bolus of the conducting medium is in contact with the one or more electrodes and extends from the one or more electrodes towards the target tissue site.

The elongate member may comprise a delivery catheter. The one or more expandable elements may comprise one or more balloons positioned on the delivery catheter.

The medical device may further comprise one or more extendable elements, the controller being further configured to advance the one or more extendable elements past a tip of the elongate member from a first site in the vessel to a second site close to the target tissue site than the first site. The one or more extendable elements may comprise a guidewire.

At least one of the one or more ports may be positioned on at least one of the one or more extendable elements. Said at least one of the one or more ports may be positioned proximate a tip of said at least one of the one or more extendable elements.

At least one of the one or more electrodes may be positioned on at least one of the one or more extendable elements. Said at least one of the one or more electrodes may be positioned proximate a tip of said at least one of the one or more extendable elements.

At least one of the one or more electrodes and at least one of the one or more ports may be positioned on at least one of the one or more extendable elements. Said at least one of the one or more electrodes and said at least one of the one or more ports may be positioned proximate a tip of said at least one of the one or more extendable elements.

The medical device may further comprise one or more fluid reservoirs containing the conducting medium.

The controller may be further configured to deliver a bolus of non-conducting medium prior to delivery of the bolus of conducting medium, the bolus of non-conducting medium flushing one or more bodily fluids from the target tissue site. The medical device may further comprise two or more fluid reservoirs, a first one of the two or more fluid reservoirs containing the conducting medium and a second one of the two or more fluid reservoirs containing the non-conducting medium.

The medical device may further comprise one or more infusion pumps, wherein the controller utilizes the one or more infusion pumps to deliver the bolus of the conducting media via the one or more ports towards the target tissue site.

The medical device may further comprise one or more generators, wherein the controller utilizes the generators to apply the energy pulses via the one or more electrodes for the therapeutic electroporation while the bolus of the conducting medium extends from the one or more electrodes towards the target tissue site.

The one or more electrodes may comprise one or more feeder electrodes and one or more return electrodes, the energy pulses being applied between respective pairs the one or more feeder electrodes and the one or more return electrodes. The one or more return electrodes may be positioned on the elongate member remote from the one or more feeder electrodes. The one or more return electrodes may also or alternatively be positioned on an additional elongate member positioned within another vessel. The controller is configured to measure impedance between the one or more feeder electrodes and the one or more return electrodes to determine when the bolus of conducting medium is in contact with the one or more feeder electrodes.

In some embodiments, a system comprises a first medical device comprising an elongate member, one or more expandable elements positioned on the elongate member, and one or more ports, a second medical device comprising one or more needle electrodes, and at least one controller. The at least one controller is configured to actuate the one or more expandable elements of the first medical device within a vessel of a subject to block fluid flow through the vessel downstream to a target tissue site, to advance the one or more needle electrodes of the second medical device into the target tissue site, to deliver a bolus of non-conducting medium via the one or more ports of the first medical device towards one or more needle electrodes of the second medical device inserted into the target tissue site to displace one or more biological fluids in a vicinity of the target tissue site, and to apply energy pulses via the one or more needle electrodes of the second medical device for therapeutic electroporation.

At least one of the one or more needle electrodes may comprise an insulated region along a length thereof and exposed electrode region at a tip thereof.

The at least one controller may comprise a first controller comprised within the first medical device, the first controller being configured to actuate the one or more expandable elements of the first medical device and to deliver the bolus of non-conducting medium, and a second controller comprising within the second medical device, the second controller being configured to advance the one or more needle electrodes and to apply the energy pulses.

The one or more needle electrodes may comprise at least a first needle electrode and a second needle electrode, and the at least one controller may be further configured to measure an impedance between the first and second needle electrodes to determine when the bolus of the non-conducting medium is in contact with the one or more needle electrodes.

In some embodiments, a method comprises delivering an elongate member of a medical device to a first site within a vessel proximate a target tissue site to be treated and actuating one or more expandable elements of the medical device to block flow of one or more bodily fluids through the vessel downstream from the first site to the target tissue site. The method also comprises advancing, via one or more ports of the medical device, a bolus of conducting medium, the bolus of conducting medium extending from one or more electrodes of the medical device, positioned at a second site, towards the target tissue site. The method further comprises applying, via one or more electrodes of the medical device, energy pulses to the target tissue site, the bolus of conducting medium extending a field gradient of the energy pulses from the second site to the target tissue site.

The second site may comprise a tip of one or more extendable elements of the medical device. The method may further comprise, prior to advancing the bolus of the conducting medium, advancing a non-conducting medium via the one or more ports of the medical device through the target tissue site to flush the one or more bodily fluids from the target tissue site.

Advancing the bolus of the conducting medium may comprise introducing the bolus of the conducting medium via the one or more ports of the medical device and introducing additional non-conducting medium via the one or more ports of the medical device to advance the bolus of the conducting medium from the second site towards the target tissue site.

Advancing the bolus of the conducting medium may comprise advancing a first bolus of the conducting medium through the target tissue site to extend from at least a first one of the one or more electrodes providing a feeder electrode for the one or more energy pulses to at least a second one of the one or more electrodes providing a return electrode for the one or more energy pulses, advancing a bolus of non-conducting medium past the feeder electrode, and advancing a second bolus of the conducting medium to a region surrounding the feeder electrode, wherein the one or more energy pulses are applied while the first bolus of the conducting medium is in a region surrounding the return electrode and while the second bolus of the conducting medium is in the region surrounding the feeder electrode.

Advancing the bolus of the conducting medium may comprise advancing a first bolus of non-conducting medium through the target tissue site to extend from at least a first one of the one or more electrodes providing a feeder electrode for the one or more energy pulses to at least a second one of the one or more electrodes providing a return electrode for the one or more energy pulses, advancing the bolus of the conducting medium to a region surrounding the feeder electrode and extending towards the target tissue site, and advancing a second bolus of the non-conducting medium behind the bolus of the conducting medium, wherein the one or more energy pulses are applied while the bolus of the conducting medium is in the region surrounding the feeder electrode.

### BRIEF DESCRIPTION OF THE DRAWINGS

Several aspects of the disclosure can be better understood with reference to the following drawings. In the drawings, like reference numerals designate corresponding parts throughout the several views.

FIG. 1 shows a catheter body of a medical device inserted and positioned in a subject with an expandable element near to target tissues in an illustrative embodiment.

FIG. 2 shows a catheter body of a medical device inserted and positioned in a subject with multiple expandable elements near to target tissues in an illustrative embodiment.

FIG. 3 shows a guidewire extending from an expandable element of a catheter body near to target tissues in an illustrative embodiment.

FIG. 4 shows a sequence of boluses of fluid introduced near to a feeder electrode on an extendable element extending from an expandable element of a medical device in an illustrative embodiment.

FIGS. 5a-5c show medical devices positioned within vessels along with expandable elements and extendable elements from which boluses of non-conducting and conducting media are delivered in illustrative embodiments.

FIG. 6 shows a return electrode positioned near receiving vasculature in an illustrative embodiment.

FIG. 7 shows an isolated guidewire body of a medical device position near to target tissues along with moving waves of maximal field gradient extending from a feeder electrode on the guidewire body in an illustrative embodiment.

FIG. 8 shows a guidewire of a medical device positioned within a vessel along with field gradients extending from a bolus of conducting media surrounding a feeder electrode on the guidewire.

FIG. 9 shows non-conducting and conducting media positioned within a vessel, along with a meniscus between the non-conducting and conducting media in an illustrative embodiment.

FIGS. 10a-10d show use of balloon-based delivery catheter medical devices for treating a target region of tissue in an illustrative embodiment.

FIG. 11 shows a balloon-based delivery catheter medical device positioned in a vessel with a guidewire extending thereof towards a target region in an illustrative embodiment.

FIG. 12 shows a catheter-based medical device from which a guidewire extends along with non-conducting and conducting media delivered from ports thereof in an illustrative embodiment.

FIG. 13 shows a balloon-based delivery catheter medical device including a guidewire with a feeder electrode at a tip thereof extending from a balloon towards a target region, along with a fluid migration vector for non-conducting and conducting media delivered from ports of the guidewire, in an illustrative embodiment.

FIG. 14 shows a balloon-based delivery catheter with a balloon positioned upstream of a bifurcation of a vessel and a guidewire extending from the balloon along one branch of the bifurcation in an illustrative embodiment.

FIGS. 15a-15c show medical devices positioned in branched vessels along with non-conducting and conducting media delivered from the medical devices in illustrative embodiments.

FIG. 16 shows a combined percutaneous and vascular approach for therapy using catheter-based and needle electrode medical devices in an illustrative embodiment.

FIG. 17 shows areas for placement of portions of medical devices for performing therapies for a target tissue in an illustrative embodiment.

FIGS. 18a and 18b shows medical devices configured for delivery of non-conducting and conducting media in synchronization with electrical pulses to feeder electrodes for performing therapeutic electroporation of target tissues in an illustrative embodiment.

FIG. 19 shows a block diagram of hardware components of a medical device in an illustrative embodiment.

FIG. 20 shows a method for operating a medical device for therapeutic electroporation of target tissues in an illustrative embodiment.

FIGS. 21a and 21b show a medical device positioned within an arterial supply of an organ including target tissues to be treated in an illustrative embodiment.

FIGS. 22a and 22b show a fractal tree structure representing a portion of microvasculature of a subject which may be accessed and treated using therapeutic electroporation in an illustrative embodiment.

FIG. 23 shows plots of cross-sectional area and velocity of fluid flow through different portions of an anatomy of a subject in an illustrative embodiment.

FIGS. 24a-24i show methods for delivery of non-conducting and conducting media between feeder and return electrodes of medical devices for performing therapeutic electroporation of target tissues in illustrative embodiments.

FIG. 25 shows application of a pulse train with asymmetric pulses of opposite polarities in an illustrative embodiment.

### DETAILED DESCRIPTION

Particular embodiments of the present disclosure are described hereinbelow with reference to the accompanying drawings; however, the disclosed embodiments are merely examples of the disclosure and may be embodied in various forms. Therefore, specific structural and functional details disclosed herein are not to be interpreted as limiting, but merely as an illustrative basis for the claims and as a representative basis for teaching one skilled in the art to variously employ the present disclosure in virtually any appropriately detailed structure. Like reference numerals may refer to similar or identical elements throughout the description of the figures.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As discussed above, various approaches have been developed to deliver energy to target tissues within a subject (e.g., for neuromodulation, denervation and/or ablation of target tissues), including catheter-based approaches that involve a deployable mechanical structure with hard, metallic electrodes that need to conform to the surface of the target tissues to be treated. As vessels narrow at the parenchyma of an organ, near to a tumor site, etc., it is challenging to reduce the size of the mechanical components and electrodes of a catheter-based medical device to reach the target tissue while also being able to maintain an effective treatment volume.

Illustrative embodiments provide medical devices configured for therapeutic electroporation of biologic tissues which overcome these and other challenges of conventional approaches. Advantageously, the medical devices described herein are configured for creating and modifying a spatial and time-varying field strength and distribution of energy delivered during such therapeutic electroporation.

In some embodiments, a medical device includes one or more expandable elements configured to seal off one or more regions of an anatomical surface, to substantially isolate a region thereof from adjacent biological fluids. The medical device may include one or more ports and delivery means for a coupling medium, such that one or more of the isolated regions may be brought into contact with the coupling medium. The medical device may further include one or more electrodes, arranged amid the one or more expandable elements, the one or more ports, and/or a guidewire coupled with the delivery catheter such that at least one of the one or more electrodes are brought into contact with the coupling medium after the coupling medium is delivered through the one or more ports. The at least one electrode is electrically connectable to one or more of the isolated regions via the coupling medium. The medical device may include a generator that is coupled with the at least one electrode to provide signals to one or more of the isolated regions via the at least one electrode and the coupling medium during use.

The coupling medium may include, but is not limited to, a fluid, a gel, a liquid, a temperature or pH transition gel, a fluid composite, or the like. In some embodiments the coupling medium may include one or more drug or other therapeutic components, provided so as to treat one or more tissue sites near one or more target regions (e.g., the isolated regions). Some non-limiting examples of such drug or other therapeutic components include a medicament, one or more tissue ablating agents (e.g., alcohol, ethanol, isopropyl alcohol, benzyl alcohol, phenol, ethanolamine, athanolamine oleate, sodium tetradecyl sulfate, a chemotherapeutic agent, combinations thereof, etc.), a therapeutic agent (e.g., a neuroblocking agent, ethyl alcohol, botulinum toxin, neurotoxin, paclitaxel, combinations thereof, etc.), a denervating agent, a sympathetic nerve specific denervating agent (e.g., ethanol, phenol, botulinum toxin, a derivative, a combination thereof, etc.), a parasympathetic nerve specific denervating agent, a neuroblocking agent, a highly specific neuroblocking agent (e.g., an agent specifically configured for blocking of a particular receptor, nerve family, etc.), an antibody drug conjugate (ADC) substance, a chemotherapeutic agent, a toxin, a neurotoxin, etc.

The ADC substance may be configured to affect the function of a region or tissue type within the vicinity of an organ, alternatively to the other tissues within the vicinity thereof. In some embodiments, the substance may include a sugar attached to a therapeutic agent to mask the therapeutic agent, such that it is taken up by the region of tissue (e.g., since it appears as a sugar, a friendly protein, etc.). Such a configuration provides a method for delivering a highly potent medicament directly to a tissue of interest (e.g., directly into a tumor), so as to enhance the bioavailability thereof, and to minimize the systemic dosage required in order to achieve significant therapeutic concentrations thereof within the region of tissue. The denervating agent may be ethanol, botulinum toxin, etc. The highly specific denervating agent may be a neural targeting chemical such as a poison, a toxin, etc.

In some embodiments, the one or more expandable elements of the medical device are formed such that, upon expansion near to one or more target regions that are to be isolated, the shape of the isolated region(s) may be reliably established even in areas of anatomical variation. The one or more expandable elements may be formed such that, upon expansion in a tubular structure, the isolated region(s) may substantially form a cylinder. In aspects, the length of an isolated region may be greater than 1 millimeter (mm), 2 mm, 4 mm, 8 mm, 12 mm, or the like.

Regions which may be accessed and/or targeted using the medical devices described herein include, but are not limited to: tubes or vessels (e.g., arteries, veins, lymphatic vessels, etc.) including bifurcated vessels, near to a bifurcation, between vessels near a bifurcation, between adjacent arteries and veins; vessels within an organ, within soft tissues, chamber walls (e.g., through the thickness of a chamber wall); into vessels within a wall of the heart; vessel entrances and/or exits to one or more chambers (e.g., of the heart or other organs or target tissues); microvasculature (e.g., driving fluid into the microvasculature, while periodically providing pulsed field ablation (PFA) signals for ablation, for drug delivery, combinations thereof, etc.); within the vasculature and/or microvasculature of a bone; within the marrow of a bone; along a vessel as and down into an organ or other target tissues; a lobe of an organ, a region within an organ, a tumor, the vasculature serving a tumor, etc.

In some embodiments, the medical device is configured to deliver (e.g., via the one or more ports) an infusion of a sequence of one or more non-conducting fluids and one or more conducting fluids, to form one or more impedance-controlled volumes, such as a conductive region downstream of the one or more expandable elements (e.g., towards the parenchyma of an organ, towards the tissues of a tumor, etc.). Advantageously, this enables targeting of tissues, such as nerves closer to a target organ (or other region of interest) with an electrode positioned out ahead of the catheter tip to engage with the conducting portion of the fluid as it is advanced towards and/or through the target tissues.

In some embodiments, the one or more expandable elements of the medical device include one or more balloons, with the target region being in, around or between one or multiple balloons. The target region may include a downstream volume, microvasculature within the walls of a vessel, one or more nerves nearby a vessel, microvasculature from a vessel supplying the target region, which is treated with extra elements (e.g., at least one of the one or more electrodes) to deliver local therapy distal to a tip of catheter. One or more of the balloons may include perforated walls, such as structured perforations, to tailor the target region. One or more of the balloons may include contoured concave regions, to allow for a fluid volume to fill around the perforations so it situates between the perforations and the tissue surface. One or more of the balloons may be a leaky balloon, where the leakage creates conductive regions around the tissues, with the conductivity defined by the nature of a conducting fluid or medium delivered therethrough.

A medical device, in some embodiments, includes one or more needles configured to perforate through walls and create a volume of conducting fluid. In aspects, the fluid may be a thixotropic fluid so as to form a controlled volume upon delivery (e.g., via one or more needles, via one or more ports, etc.). The fluid in some embodiments is configured to be both delivered from and recovered by the medical device. In some embodiments, fluid may be provided from the medical device in multiple forms, including a first form configured so as to electrically isolate fluids along a vessel and a second form configured so as to electrically connect tissues along a vessel to form a shape along which an isoelectric surface can be formed, such that an electric field vector created along the region will be substantially orthogonally to the outer surface of the region and maximized for adjacent tissues. The use of multiple fluids, including fluids in the first and second forms, may be used to help control field formation and prevent unwanted field gradients (e.g., except in the directions needed for therapy).

The medical device may include means for measuring the impedance of the tissue interface, where such measurement may be performed prior to and between delivery of therapeutic energy thereto. Such an approach may be used to limit the potential for arching and/or barotrauma in the vicinity of target treatment sites. In some embodiments, the medical device includes one or more sensors configured to determine impedance between a target treatment region and a return path. The impedance may be used to determine the overall area or extent of the fluid electrode, and may be used to dictate or control the energy delivered to the fluid electrode during one or more PFA or other therapeutic pulses. The impedance may also be used to monitor for changes in the impedance of nearby tissues as pulse trains are delivered (e.g., to determine the changes from pulse to pulse in the train, during breaks between pulse trains, combinations thereof, etc.). The medical device may also or alternatively include one or more sensors configured to determine the local temperature near the target treatment region, such that the temperature may be used as feedback to limit and/or regulate delivery of therapy to the target treatment region. The medical device may further or alternatively include one or more sensors configured to tailor the energy delivery in a pulse (e.g., to the level needed to establish a therapeutic field gradient in the tissues adjacent to the target treatment region).

In some embodiments, the fluid delivered via the medical device is cooled to minimize thermal load on adjacent tissues. One or more of the fluids, in some embodiments, have high thermal conductivity to prevent overheating of the adjacent tissues. The fluid may also or alternatively be tailored to gel in the presence of the target tissues, or in presence of water such that it may be provided in a low viscosity form so as to quickly pass through the catheter and gel when it comes into contact with the surrounding fluids (e.g., to form a biodegradable skin on a balloon and establish a therapeutic electrode surface).

In some embodiments, the one or more expandable elements of the medical device provide at least one balloon having multiple chambers, with the balloon chambers being divided and filled with various fluids to as to shape the fluid electrode from which therapy will progress, while providing durable electrical isolation around the target treatment region so as to minimize energy loss into the vessel, to prevent strong fields outside of the intended treatment region, so as to infuse various fluids into the microvasculature of the vessel into which the balloon is placed, etc.

The medical device may include means for isolating pulses, so that the energy from a pulse does not break down into the surrounding fluids until reaching the intended target tissues. The medical device may also include means for isolating pulses such that the capacitance of the structure is minimized between a coupled generator and the target delivery region. Such means may include, by way of example, use of a low dielectric permittivity fluid in adjacent chambers and/or sequentially delivered so as to contain a bolus of conducting fluid along the target vessel. Such means may also or alternatively include the use of fluids that change permittivity with temperature, so as to block current flow through them above a particular temperature. Such fluids may include, but are not limited to, biocompatible and biodegradable poly-ε-caprolone (PCL) and polylactide (PLA), as well as their nanocomposites, with low impedance as measured in radiofrequency and millimeter (MM) wave bands. Such means may further or alternatively include the use of fluids that change their dielectric properties when exposed to light, where the medical device includes a light source to quickly switch material behavior during use.

In some embodiments, the use of multiple chamber balloons in the medical device enables better shaping of the fluid electrode region near the target treatment region. Dimples in the balloons may be configured to hold the fluid and distribute charge to support a short, high intensity electrical pulse into tissues adjacent thereto. One or more of the balloons may also include a thin film walled balloon with conductive regions to support establishing a field with very little gradient along the edge of the region, such that the maximal gradient is orthogonal into the adjacent tissues.

The medical device, in some embodiments, is tailored to minimize impedance to pulses delivered therefrom via the coupling fluid. The pulse delivery timeframe may be adjusted to meet the high frequency impedance spectrum of the coupling medium. The coupling medium may be provided with a carefully controlled molarity, such that there is a critical oscillation frequency where the capacitive to inductive frequency regime of the coupling medium transition jumps from a high to a low frequency region in the coupling medium. The electrical impedance of the fluid may be minimized at this critical frequency. Alternatively, the concentration of ionic species in the fluid may be tailored to the pulse period of the applied electrical pulses to minimize the impedance of the solution over that timeframe. The critical frequency may change from between 100 kilohertz (kHz) to 1 megahertz (MHz) over a molarity change of between 1 millimolar (mM) and 500 mM concentration of the coupling medium. In aspects, solutions with divalent, trivalent, and/or tetravalent ions may be used to achieve a lower critical concentration than solutions with monovalent ions.

In some embodiments, the one or more expandable elements of the medical device may be used to temporarily stop the flow of a biological fluid, such as blood, to one or more regions of the target tissues. Such capability may be used for various purposes, including but not limited to holding one or more coupling media at the intended location, flushing the target region of one or more bodily fluids (e.g., blood, lymphatic fluid, bile, pancreatic fluid, urine, etc.), controllably delivering a bolus of coupling media to the target tissues, controllably creating the volumetric electrode at the intended therapy delivery site, controllably establishing and propagating a wavefront of maximal field gradient through vasculature and microvasculature of the target tissues, flushing the target tissues with one or more drugs and/or ionic species, so as to prepare them for therapy, temporarily starving the target tissues of oxygen and nutrients prior to delivery of therapy, a drug, a toxin, or the like, etc. In terms of starving the tissues of oxygen and nutrients, such a process may make the local cells more receptive to ablative therapy, lowering the threshold ablative field within the tissues, making the tissues more receptive to drug or toxin uptake, etc. The one or more expandable elements of a medical device, in some embodiments, may be reversibly expanded and contracted to flush fluids through the target region, to re-establish blood flow back to the target tissues after therapy, etc.

In some embodiments, pulse delivery is combined with one or more constituents in the coupling medium so as to increase the therapeutic effect thereof. Some non-limiting examples include using repetitive pulses to open pores in cell membranes, using asymmetric pulse trains to increase membrane polarization, increasing medium conductivity, etc., which may be used to improve the uptake of an included drug, toxin, medicament, therapeutic substance or other constituent in the fluid, including constituents which may disrupt local metabolic processes, such as sodium (Na+), potassium (K+), magnesium (Mg2+), and calcium (Ca2+) in the coupling medium, or the like.

In some embodiments, a system may include means for measuring cardiac ventricular activity, the system including an algorithm configured to apply pulses in synchronization with the measured activity so as to prevent ventricular fibrillation or other proarrhythmic effects during therapy. The system may include an algorithm to tailor the pulses and pulse trains to minimize and/or eliminate local skeletal muscle contraction and pain associated with application of therapeutic pulses. Such pulse characteristics may be adjusted to a period of less than 100 microseconds (μs), less than 20 μs, less than 10 μs, less than 5 μs. less than 2 μs, less than 1 μs, less than 0.4 μs, or the like. The pulse width may be variable between 0.2 μs and 10 μs throughout the pulse train. Each pulse may be formed as preferably an asymmetrical bipolar signal, the asymmetric bipolar signal changing in polarity throughout the pulse train, and the pulse spacing may be on the order of less than 1,000 milliseconds (ms), less than 100 ms, less than 5 ms, less than 1 ms, less than 500 μs, less than 100 μs, less than 25 μs, less than 10 μs, less than 2 μs, or the like. Such pulse trains may be used to limit the need for general anesthesia/paralytics and intubation of patients prior to therapy.

The asymmetrical pulses may be applied in reverse polarity throughout the pulse chain, to minimize changes of muscle contraction while increasing the produced ablation volumes. The asymmetrical pulses may be adjusted throughout the train such that charge delivery is initially biased in a first polarity (e.g., a positive polarity), then in a second, opposite polarity (e.g., a negative polarity), with the timespan of the variation between first and second opposite polarities changing on a scale that is sufficiently rapid so as to minimize long-term charging of tissues, but yet long enough so as to maximize local electroporation of nearly tissues. Such an approach may be considered to introduce a changing polarity bias to the pulse train. The polarity bias may change at a rate of greater than 100 Hz, greater than 1,000 Hz, 10,000 Hz or the like.

In some embodiments, the amplitude of the bias may be adjusted in real-time during pulse delivery to minimize long-term charging of remote tissues from the treatment site. The amplitude of the bias may be adjusted from +/−100% (e.g., essentially a monophasic pulse train), through to 0% (e.g., a balanced biphasic pulse train).

In some embodiments, the amplitude of the bias may be adjusted based on charge measurements made from one or more remote sites on and/or in the body of the subject (e.g., from a remote internally placed electrode, from a patch electrode on the body, etc.). The bias may be adjusted so as to prevent stimulation of nerves and/or muscles in such tissues, thus potentially obviating the need for general anesthesia and/or application of paralytic agents during a procedure.

Using an asymmetric pulse train, ablation volumes may be increased by a factor of at least 2×, and often up to 5×, that of a symmetric pulse train. Asymmetric pulses imply a biphasic pulse where a positive and negative amplitude and/or pulse width may be different from each other. In aspects, the pulse train may be configured such that asymmetry of the pulse train changes from primarily longer positive polarity pulses to primarily longer negative polarity pulses over the overall delivery period of the pulse train. The frequency with which the asymmetry shifts from positive to negative and back may be on the order of greater than 1 kHz, greater than 10 kHz, greater than 100 kHz, or the like. The shifting asymmetry throughout the pulse train allows for the application of asymmetric pulses to the tissue (e.g., thus potentially lowering the ablation thresholds thereof), while providing short-term charge asymmetry to the target tissues, maintaining a long-term neutral overall energy delivery and minimizing long-term charge imbalance around the treatment site.

Combined with the device configurations herein, tailoring of the electrically applied pulses to focus on ultra-high frequency pulse application may significantly improve the field gradients around the intended target tissues, while limiting procedural times and risk to the patient during such procedures. In particular, tailoring of pulse parameters to the frequency response characteristics of the coupling medium and/or a combination of the coupling medium and therapy sites may be used to preferentially deliver maximal field gradients to the target locations.

Electrical pulses may be applied in such a manner so as to establish field gradients in the target tissue of greater than 500 volts per centimeter (V/cm), greater than 700 V/cm, greater than 1,500 V/cm, greater than 4,000 V/cm, or the like. The pulses may be provided as bipolar pulses, and may be provided as asymmetrically bipolar pulses to maximize local charge fluctuations in adjacent tissues, thus potentially lowering the therapeutic threshold in such tissues and more easily establishing irreversible changes with minimal input energy.

The medical device may be configured for a coordinated and controlled propagation of the coupling medium (e.g., a substantially conducting fluid, a combination of adjacently delivered substantially conducting and non-conducting fluids, etc.) through the target anatomy during periodic application of pulses (e.g., PFA pulses) thereto. In this way, the front of the maximum field gradient can propagate throughout the target tissue maximizing the therapeutic effect at sites throughout the target region and ensuring full coverage of therapy to the target region of interest. Such an approach would preferentially follow the natural pathway of the blood supply through the target region, thus optimally targeting therapy through to the local arterioles and capillaries of the target tissues.

By combining the fluid propagation and pulsation, a much lower pulse voltage may be suitable to establish therapeutic thresholds in the target tissues. Thus, instead of having to hit the entire target volume from an electrode situated somewhat remotely thereto, the fluid propagation into the target tissue may effectively extend the location of the electrode nearer to the target tissues. By propagating the coupling fluid through the target, the electrical field generated with each pulse can establish a front of the maximal electric field that can ensure thorough treatment is applied with minimal energy requirements and collateral damage to adjacent tissues.

A return path may be established by one or more secondary electrodes, via a body patch-based pathway, etc. In such cases, the area of the return path may be preferably several times greater in area than the primary electrode and/or virtual electrode site so as to minimize local field gradients in these regions with tissues that are not intended to be treated. In such situations, the applied waveforms are preferably of short pulse duration, bipolar and asymmetric so as to prevent widespread muscle contractions in the subject during pulse delivery and to effectively produce clinically relevant ablations from a single electrode source. The degree of pulse asymmetry and varying of the asymmetry over the pulse train may be used to accomplish this goal while maintaining a wide region of therapeutic effect in the target tissues. The return path may be established by one or more secondary electrodes, positioned locally to the target tissues, such that the pulses are delivered locally only to the target region.

In some embodiments, the medical devices described herein may be used in approaches for applying treatment to a tumor. In such cases, the coupling medium may be provided to a location such that the field through the tumor is maximized during application thereof.

The coupling medium may include calcium salts, introduced so as to lower the barrier for treatment of coupled tissues. Local infusion of hypertonic saline into the vasculature of the target tissues may be used to increase conductivity of tissues in the vicinity thereof, and thus help to maximally distribute field around and across the target tissues particularly in cases where the electrical conductivity of the target tissues is not as high as the surrounding tissue.

In some embodiments, the coupling medium is provided in the form of a bolus of fluid to shape the incoming pulse to create a virtual or conformal electrode for delivery of PFA pulses to target tissues. This approach allows for creation of various volumes of fluid, and for purposefully directing signals between them to wave shape in the vicinity of the target tissues.

In some embodiments, such a bolus may be provided through needle delivery mechanisms.

Generally speaking, the bulk resistivity of tissues is often between 500-1000 ohms-centimeter (ohm-cm) for muscle, liver and lung, while reaching 1500-5000 ohm-cm for fat tissues. Blood is often more conductive, roughly around 100ohm-cm. and intercellular fluid is roughly around 60 ohm-cm. Physiological saline (0.15M NaCl) is about 70 ohm-cm at low frequency and varies with salt content demonstrating strong changes in impedance over the frequency range of 1 kHz to 1 MHz. In some embodiments, a hypertonic saline solution is used to create a substantially more conductive region for applying therapeutic pulses. For example, a hypertonic saline solution of roughly 10% by weight may maintain a bulk resistivity of around 12 ohm-cm. The U.S. Food and Drug Administration (FDA) has approved 3% and 5% hypertonic saline for use in hyponatremia and increased intracranial pressure applications. Investigational studies have used infusions of 20% hypertonic saline to treat severe intracranial hypertension, thus demonstrating that small boluses of such concentrates are safe and clinically relevant.

In some embodiments, hypertonic saline (or optionally other salts), with concentrations of greater than 2%, greater than 4%, greater than 9%, greater than 18% or the like, may be used for the coupling medium to increase local conductivity to 10-20× greater than blood, and >100× greater than the adjacent tissues. This may allow the coupling fluid to better distribute charge and establish the desired therapeutic field than would be achievable with metal electrodes. The coupling medium may include one or more divalent or trivalent ion constituents so as to further improve electrical performance for the intended delivery of electrical pulses to the target tissues.

One or more of the ports of a medical device may be used to draw back the coupling medium, drugs, toxins, combinations thereof, etc. after the delivery of therapy, to minimize residual material left at the treatment site after completion.

In some embodiments, the vessel into which the coupling medium (e.g., a hypertonic solution, conducting solution, combinations thereof, etc.) is provided may be momentarily closed off from the blood supply, so as to controllably maintain the conductivity of fluid in the vessels around the target tissues with a minimal bolus. In such cases, the coupling medium allows for extension of the electrode beyond the device tip, to extend field application towards the intended tissues.

A medical device for treating a region of target tissue may include an elongate catheter, the elongate catheter shaped with a tip so as to be delivered into the local arterial supply of the target tissue. The medical device also includes one or more electrodes, the one or more electrodes attached to the tip of the catheter or an extendable component thereof (e.g., a guidewire). The one or more electrodes are positioned along the outer surface of the catheter tip (or the extendable component thereof) to couple electrically within a region surrounding the catheter tip (or the extendable component thereof). The medical device may further include one or more ports configured to deliver one or more fluids into a region surrounding at least one of the one or more electrodes. The medical device may further include one or more expandable elements, the one or more expandable elements being configured (when expanded) so as to controllably shut off blood flow to the local arterial supply during expansion thereof.

At least one of the one or more electrodes may be extendably positionable ahead of the catheter tip (e.g., via an expandable element of the medical device such as a guidewire). The medical device may include one or more reservoirs, where the reservoirs are coupled with the ports of the medical device. At least a first one of the one or more reservoirs may accommodate a substantially highly conducting medium, deliverable through at least one of the one or more ports. At least a second one of the one or more reservoirs may accommodate a substantially poorly conducting medium, deliverable through at least one of the one or more ports (e.g., which may be the same as or different than the ports used for delivery of the substantially highly conducting medium).

In some embodiments, the guidewire may include an insulated sheath, a conductive core (e.g., a wire core), and an exposed electrode (e.g., a pulse feeder electrode) at the tip thereof. The guidewire may also include a hollow core for delivery of one or more fluids from a port positioned near the tip thereof. The guidewire may be configured such that it is steerable to the arteries feeding the target tissues. The guidewire may be made sufficiently small and flexible enough to reach a vessel with diameter smaller than 2 mm, smaller than 1.5 mm, smaller than 1 mm, smaller than 0.5 mm, smaller than 0.25 mm, or the like. The diameter of the guidewire tip may be constructed to have diameter smaller than 1 mm, smaller than 0.5 mm, smaller than 0.25 mm smaller than 0.2 mm, or the like.

The medical device may include or be coupled to a generator, configured to accommodate delivery of pulses (e.g., PFA pulses) through the generator to at least one of the one or more electrodes during use. The medical device may also include or be coupled to one or more infusion pumps, each infusion pump configured to deliver one or more of the coupling media to one or more of the ports. The one or more infusion pumps, either individually or in concert, may include or be configured to implement a sequencing algorithm, configured to deliver different fluids in order to create substantially conducting and non-conducting regions in the vicinity of the target tissues during use. The combination of the fluid regions are usable to shape the region of therapy without excessive power delivery and/or delivery of therapy to regions outside of the intended target tissues.

In some embodiments, the medical device includes a guidewire, the guidewire being configured with one or more electrodes at the tip thereof. The one or more electrodes are exposed to provide an electrically coupled interface with the surroundings thereof. The medical device may further include means (e.g., an infusion pump) for controllably delivering the coupling medium to the guidewire tip, and subsequently from the guidewire tip into the vicinity of the target tissues, along with means (e.g., one or more expandable elements) for stopping blood flow in the vicinity of the target tissues so as to temporarily hold the coupling medium in the vicinity of the one or more electrodes. In some embodiments, a first coupling medium is used to create an electrically conductive medium around at least one of the one or more electrodes (e.g., “feeder” electrodes at the guidewire tip), so as to extend the effective electrode area to the surrounding tissues and to maximize the field gradients around the tissues adjacent thereto. A second coupling medium may be used to establish the boundary of the conducting medium with a substantially non-conducting adjacent region.

In some embodiments, a system including the medical device provides means (e.g., a generator and infusion pump) for controllably advancing fluids through the vessels adjacent to the target tissues in combination with delivery of electrical pulses to one or more of the electrodes. Such an approach may be suitable for propagating the maximal field gradient through the target tissue region during a therapy. The generator may be coupled to deliver electrical pulses, through the one or more electrodes and the coupling medium, to the adjacent target tissues thus enabling maximization of the field gradients in the target tissues with a minimal applied voltage and energy level. In some embodiments, the system may be configured to slowly advance the coupling medium out through the target tissues, thus expanding the field of effect from pulse to pulse so as to maximize the field gradients throughout the tissues during therapy. The coupling medium may include a contrast agent for visualization under an imaging modality. Various imaging systems, such as X-ray, computed tomography (CT), magnetic resonance imaging (MRI), ultrasound, positron emission tomography (PET), etc. may be used. The imaging may be used to ensure the coupling medium and/or non-conducting medium are in the correct location prior to the application of therapy.

The medical devices described herein may be used in various methods for treating target tissues. A method for treating target tissue may include, for example, delivering one or more electrodes to a site within the local blood supply of the target tissue. The one or more electrodes, also referred to as feeder electrodes, may be part of a medical device, such as on a guidewire that extends from a delivery catheter to a region proximate the site within the local blood supply of the target tissue. The delivery catheter may include one or more expandable elements (e.g., one or more balloons) that when expanded block the flow of blood to the target tissue. The method may also include delivering and holding a conductive coupling medium into a subregion of the local blood supply of the target tissue, where the subregion is a region proximate the feeder electrodes. The conductive coupling medium may be delivered via one or more ports of the medical device (e.g., on balloons or other expandable elements of the delivery catheter, on the guidewire that extends from the delivery catheter). The method may further include providing one or more electrical pulses to the target tissue via the feeder electrodes and the conductive coupling medium.

The coupling medium, in some embodiments, is delivered slowly along with the electrical pulses to expand the effective electrode volume in the vicinity of the target tissue. Such an approach may be used to move an electrode front through a tumor (or other target tissue region) so as to maximally treat the region while minimizing collateral damage and risk to adjacent tissues.

Methods for treating a region of target tissue in a subject may use a catheter-based medical device, where a catheter is delivered into the regional blood supply of the target tissue. The catheter includes one or more expandable elements, one or more fluid delivery ports, and one or more electrodes. The one or more expandable elements are configured to substantially isolate one or more regions around the medical device and/or downstream from the medical device, and/or to stop or slow blood flow to the target tissue when expanded. The fluid delivery ports are configured so as to deliver a coupling fluid to a region formed by the expandable element, the one or more electrodes (e.g., feeder electrodes) arranged such that upon delivery of the coupling fluid, the feeder electrodes can be electrically coupled to the surrounding tissues.

The coupling medium may be used to temporarily extend the reach of the feeder electrodes, and to optimize field gradients throughout the target tissues (e.g., potentially even along arteriole and capillary walls, etc.). This allows for maximization of therapy with a simple mechanical catheter construction, even in the presence of challenging anatomical features. In some embodiments, the coupling medium may include a range of micro and/or nano sized electrically conducting particles. Such particles may be constructed, for example, from one or more biodegradable materials. The one or more biodegradable materials may be plated with one or more conducting metallic elements, such as magnesium, a magnesium alloy, gold, iron, an iron alloy, zinc, a zinc alloy, or the like. Such particles may be sufficiently small to pass down into the arterioles of the target region, but large enough so as to not pass through the capillaries. The particles may be loaded with one or more drug elements, such as one or more cancer-treating drugs or other types of therapeutic substances as described herein. The coupling medium may include one or more electrically and/or ionically conducting fluids such that, upon delivery into the target tissues, the liquid may come into contact with the conducting micro/nano particles. Such a configuration may be advantageous to create a temporary electrode as near as possible to the target tissues, and to increase the electrical conductivity therein as much as possible.

In some embodiments, the coupling medium and/or a substantially non-conducting medium may include one or more biodegradable microparticles such as Gelfoam® for embolizations, polyvinyl alcohol particles, N-butyl-cyanoacrylate, trisacryl microspheres, or the like. Such substances may be suitable to form a temporary occlusive effect, with recanalization of the vessel occurring in a few weeks. The microparticles may be plated with thin layers of one or more conducting metallic elements, such as magnesium, a magnesium alloy, gold, iron, an iron alloy, zinc, a zinc alloy, or the like.

The local field may be generated from the temporary electrode formed by the coupling medium and an influx of charge fed by the one or more feeder electrodes. A return path may be provided by one or more local electrodes, an electrode placed onto the catheter body, an electrode positioned into the vasculature of the subject, one or more electrodes placed within an adjacent vessel, one or more electrodes placed within one or more veins served by the corresponding arteries in which the feeder electrode has been placed, an indifferent electrode placed onto the body of the subject or a fluid bath applied to one or more body regions of the subject, etc.

The coupling medium may be delivered through a local port just proximal to the position of the feeder electrode. In one non-limiting example, the catheter body may include multiple ports, such as one or more first ports coupled to a reservoir of a substantially non-conducting medium and one or more second ports coupled to a reservoir of the coupling medium.

The medical devices described herein may be used for performing various actions, including identifying a target region in a subject, positioning the one or more feeder electrodes of the medical device near to the identified target region, and coupling a return electrode to the subject. The medical device may be further used to block blood flow through the identified target region (e.g., via expansion of one or more expandable elements thereof), and to deliver fluids to the identified target region. The delivery of fluids may begin with delivering (e.g., via one or more first ports of the medical device, from one or more first reservoirs that are part of or which are coupled to the medical device) a substantially non-conducting fluid to the identified target region, displacing the blood therein. The delivery of fluids may continue with delivering (e.g., via one or more second ports of the medical device, from one or more second reservoirs that are part of or which are coupled to the medical device) a coupling medium into the identified target region, such that the coupling medium contacts the one or more feeder electrodes and extends therefrom (e.g., creating a fluid electrode that can access smaller parts of the anatomy than the one or more feeder electrodes). The first and second ports may be provided at substantially the same location to each other or at different locations from one another. Similarly, the first and second reservoirs may be configured to provide the same fluid or a different fluid as one another. Subsequent to delivery of the coupling medium, the medical device may be used to deliver one or more pulses (e.g., impedance measurement pulses, stimulation pulses, PFA pulses, etc.) between the one or more feeder electrodes and the return electrode to apply energy to the tissues in the target region. Such initial pulsation may be used to assess the coupling impedance therebetween prior to the introduction of the coupling medium and delivery of the therapeutic pulses.

The return electrode may be positioned locally near to the target region or may be positioned within the vasculature near the target region. An additional coupling medium may be provided which is in contact with the return electrode (e.g., to create a fluid electrode in a manner similar to the fluid electrode created by the coupling medium that is in contact with the one or more feeder electrodes). The return electrode may alternatively be applied to the body of the subject (e.g., as a patch). The return electrode may also or alternatively be provided by a fluid path. The substantially non-conducting fluid may be delivered from the medical device such that the substantially non-conducting fluid is positioned substantially between the one or more feeder electrodes and the return electrode.

In some embodiments, prior to application of energy, tissues in the target region may be perfused with a substantially non-conducting fluid. Such application may be provided from one or more ports of a medical device (e.g., a catheter or micro-catheter) that is positioned within the arterial supply of the target tissues, the blood flow through which may be blocked by the expansion of one or more expandable elements (e.g., one or more balloons) or held with a local vacuum formed by a stopcock on the medical device to temporarily and controllably hold these fluids in place during pulse ablation with a somewhat non conducting fluid first. The introduction of the substantially non-conducting fluid can be used to temporarily displace blood from the target region. Such an approach may be advantageous to displace substantially conducting media (e.g., blood, interstitial fluid, etc.) from the tissues in the target region, and may allow for more evenly distributed field application through the tissues during energy delivery steps of a therapy.

After introduction of the substantially non-conducting fluid, a controlled bolus of the coupling medium may be introduced and advanced through the tissues in the target region, in some cases in synchronization with pulsed energy delivery, so as to establish a maximal field gradient along the boundary of the coupling medium in the arterioles and capillaries of the target region. Such an approach may be suitable to maximize field gradients in all the tissues in the target region as the coupling medium is advanced therethrough. This may provide a mechanism to maximally treat the tissues with as small of an applied field as possible. In some embodiments, the introduction of the coupling medium in conjunction with the substantially non-conducting medium may help to maximize and evenly distribute the field gradients throughout all the tissues of the target region during a pulsed energy delivery procedure. The combination of maximizing and evening out the local field distribution and propagating the field wavefront through the target tissues may be used in some embodiments to achieve tissue treatment with substantially lower applied PFA voltages, lower overall energy requirements, and substantially fewer far field effects. This can potentially help in further reducing muscle contraction, and thus alleviate the need for paralytics during a procedure. Such an approach may allow for naturally maximized field gradients within the target tissues while minimizing the gradients in fluids surrounding the larger vessels and far field tissues.

In some embodiments, the field gradients around the boundary of the coupling medium may be maximized, such that the fields can cause therapeutic effects in tissues within a distance of less than 10 mm, less than 5 mm, less than 2 mm, less than 1 mm, less than 500 um, less than 200 um, or the like. As the boundary is pushed through the microvasculature of the target tissues, all cells therein will come within a short distance of the fluid boundary.

In some embodiments, the combination of media (e.g., the substantially non-conducting medium and the substantially conducting medium) could be applied to PFA procedures on the whole. In one non-limiting example, a target region is identified, blood flow to the region is temporarily stopped, a non-conducting fluid is perfused through the target region, and then a field may be applied throughout the region. The presence of the non-conducting fluid may help to maximize field gradients in the tissues within the target region and achieve therapeutic effect with minimal energy delivery and as uniformly as possible through the target region. Thus, a fluid perfusion pre-step may be used to improve therapeutic effectiveness of a more traditional field-based ablation procedure, such as when energy is delivered to the region from locally placed needles, from an endoscopically placed electrode, or the like.

The substantially non-conducting medium may include one or more non-ionic contrast agents to image the target region. The substantially non-conducting medium may also or alternatively include one or more drug elements so as to prepare the tissues in the target region for therapy, such as to increase the field sensitivity of the tissues in the target region to an electric field (e.g., decreasing the threshold field needed to initiate electroporation in the tissues in the target region), to provide a toxic drug to be infused into the tissues in the target region prior to and during an electroporation process, etc. The substantially non-conducting fluid may further or alternatively include one or more smooth muscle paralytics, an ion channel blocker, calcium channel blockers (CCBs), a nitrate, dihydropyridines (e.g., nifedipine, amlodipine, felodipine, etc.), phenylalkylamines (e.g., verapamil, etc.), modified benzothiazepines (e.g., diltiazem, verapamil, diltiazem, nifedipine, etc.), amlodipine, benidipine, a vasodilator, or the like.

A system may include a return electrode, where there return electrode may be positioned in a vessel nearby to the target region (e.g., a receiving vein near to the target region). In some embodiments, the return electrode is placed in a vein coming directly from the target region. In such cases, as the coupling medium is progressed through the target region, an impedance between the one or more feeder electrodes and the return electrode may be used to gauge the depth of penetration. When the impedance drops (e.g., when the coupling medium has reached the return electrode), the therapy is concluded.

In some embodiments, the medical device may be used to remove at least a portion of the coupling medium and/or the non-conducting medium after completion of a therapeutic pass through the target region, optionally repeating therapeutic passes for further treatment. The medical device, for example, may apply suction or vacuum force to one or more of its ports to remove the coupling medium and/or the non-conducting medium.

A system in some embodiments may include a fluid control device that is placed at a first site, where the first site is substantially upstream from a second site (e.g., a target site where the target tissues are located). The medical device with the one or more feeder electrodes and/or the ports from which the coupling medium is deliverable may be placed at one or more additional sites, the one or more additional sites being downstream from the first site but upstream from the second site where the target tissues are located. The coupling medium may be introduced from the ports of the medical device at the one or more additional sites, the coupling medium being advanced through to the second site where the target tissues are located while providing electrical pulses from the one or more feeder electrodes. In some embodiments, impedance between the one or more feeder electrodes and the return electrode is sensed to determine when the one or more feeder electrodes and/or the return electrode is in contact with the coupling medium. The impedance measurements may be used to determine an effective volume and/or area of the coupling medium during a procedure.

In some embodiments, the system may include one or more sensing electrodes (e.g., which may be on the catheter or guidewire of the medical device having the one or more feeder electrodes and the ports from which the coupling and non-conducting medium are delivered) which are used to identify the arterial branches associated with target tissues, and to select the first site, the second site and the one or more additional sites accordingly. The arterial branches may be sufficiently large such that the first and second sites may be collocated with each other. In other aspects, the first and second sites may be separated by considerable distance in order to provide effective access to the target tissues. The coupling medium and substantially non-conducting medium may be advanced in conjunction with one another to position and/or confine the movement of the coupling medium to the target tissues and the feeder electrode.

In some embodiments, the one or more feeder electrodes and the one or more return electrodes may be constructed from a metal or an alloy thereof. The electrodes may be constructed from a three-dimensional (3D) structure, such as a microporous material, a dendritic forest-shaped electrode, etc. In such configurations, the effective impedance of the electrodes may be dropped by one, two, or more orders of magnitude during use.

The system may include means (e.g., one or more expandable elements, a stopcock coupled with a local port, etc.) for blocking the arterial supply to target tissues, means (e.g., one or more ports) for advancing a substantially non-conducting medium through the target tissues, and one or more needle electrodes configured for insertion into the peripheral boundary of the target tissues. The system may be configured to apply electrical pulses between the needle electrodes (or between one or more of the needle electrodes and one or more separate return electrodes). The one or more needle electrodes may be configured with ports for introducing a coupling medium into the adjacent tissues, and the non-conducting medium may be moved so as to control the boundary and evolution of the coupling medium during therapy.

The systems and medical devices described herein overcome various challenges of conventional approaches, including in locating and reaching the arterial supply for a tumor (or other target tissues). Locating the arterial supply may use imaging elements on the medical device, the imaging elements configured for CT, MRI or other suitable types of imaging of a target site. The arterial supply for the tumor (or other target tissues) may be reached using medical devices having a delivery catheter (e.g., with one or more expandable elements providing a balloon or micro-balloon catheter) and a guidewire which extends therefrom (e.g., where the guidewire includes one or more feeder electrodes). The necessary field is created through the tumor or other target tissues through delivery of non-conducting and coupling mediums from the medical device (e.g., from ports on the medical device, which may be located on the balloon or other expandable elements thereof, on the guidewire, combinations thereof, etc.). The non-conducting and coupling mediums may be fluids which are passed through the arterial supply of the tumor or other target tissues while applying PFA signals (e.g., to the one or more feeder electrodes). Advantageously, the systems and medical devices described herein can limit or eliminate any over treatment of tissues adjacent to the tumor or other target tissues by confining the treatment to the target tissues through controlled fluid movement of the non-conducting and coupling mediums, using the balloon or other expandable elements to stop or displace local blood flow, etc.

The systems and medical devices described herein can also avoid or prevent complications that have negatively impacted needle-based PFA approaches, including but not limited to: reducing or eliminating bleeding (e.g., which is automatically overcome as no tissue is cut near the target region); avoiding bile duct rupture (e.g., which is automatically overcome as no tissue is cut near the target region); avoiding stenosis of vasculature or bile ducts (e.g., as no tissue cutting near the target region is necessary); reducing or eliminating residual pain (e.g., as there is the option to treat nerves as part of a procedure, where the tumor and nerves are treated together or optionally separately); avoiding sepsis (e.g., which is automatically overcome as no tissue is cut through to get to the target region); reducing or eliminating muscular contractions (e.g., which may be avoided through the inclusion of vasodilators in the coupling medium); reducing or eliminating electrocardiogram (ECG) synchronization issues; etc.

The systems and medical devices described herein can also utilize mono-electrode treatment profiles, which may be easier to implement than approaches which utilize two or more electrodes. Further, bipolar high frequency pulses may be used while minimizing far field gradients as described herein. The systems and medical devices described herein may also remove the need for general anesthesia and neuromuscular paralysis to avoid skeletal muscle contractions and also minimization contractions generally. Further, an optimized irreversible electroporation (IRE) ablation protocol is enabled using the systems and medical devices described herein, which advantageously avoids muscle contraction, pain, arcing, and bubble formation. Arcing and bubble formation may be avoided, for example, through the use of bipolar high frequency pulses which the fluid electrode will minimize. Further, the systems and medical devices described herein can be used in treatment of various cell types and orientations of cardiac fibers, with orientation dependence and areas of reversibility being dependent on the treatment zone. The maximum treatment zone, for example, may travel along the boundary of the coupling medium, thus getting all the way down to the capillary level and into the tissues themselves can be achieved without cutting any tissue.

In some embodiments, the systems and medical devices described herein may be used to enter and treat lymphatic vessels. Embolic microspheres (e.g., conducting spheres) may be used as part of the coupling medium for treatment as well.

Electric fields provide the principal therapeutic mechanism for biologic cell membrane modification. Electric fields across cell membranes result in membrane permeability, which is generally proportional to field strength and temporal duration of membrane-field exposure. Electric field strength may be measured in volts per meter (V/m), where one V/m is the electrical potential difference of 1 volt (V) at two points separated by one meter. Electric flux intensity measures may also be used. Electroporation may be ablative and/or therapeutic, allowing drugs or biomolecules to cross the cell membrane and interact with the cytosolic components and the nucleus. Electroporation may also initiate cell death if membrane pores are large enough and present long enough to allow intracellular and/or nucleus death. The systems and medical devices described herein enable creation and modification of electric fields in three spatial dimensions, and also enable application of time dependent electric field strength variation. The flexibility of these methods may be used to optimize field strength (e.g., in space and time), and result in vastly improved therapeutic effects. Such therapeutic effects may entail optimizing effective formation of pores in the target cells, affecting the size of the pores formed the cells, increasing the number of pores formed in the cell walls, and/or lengthening the time that pores remain open after application of the fields for either therapy or toxicity (e.g., resulting in degraded cell function or cell death).

Electric fields may be delivered to target tissues using spatial field shaping, allowing optimal field strength matched to create maximal therapeutic or toxic effect. The temporal field changes are independent of spatial changes, permitting time-varying electric fields of optimal shape. Biologic and medical applications may require therapy at a multiplicity of internal bodily sites, with different tissues to be treated. Electric fields for electroporation and other therapy must be delivered to various target sites within the body. Thus, the medical devices described herein provide catheter systems for traversing the required paths, with the catheter systems including one or more electrically conductive wires. Because the target biologic tissues may be of irregular 3D shapes, optimal delivery requires electric fields that can conform to and/or encompass the target tissue.

In some embodiments, systems and medical devices use one or more feeder and return electrodes (possibly along with various sensing electrodes as described elsewhere herein), where such electrodes may have a multiplicity of components having opposite polarity (e.g., positive and negative). In some embodiments, electrodes of 3D configuration are used, where the feeder electrodes are, for example, positioned at the terminus (e.g., the distal tip) of a guidewire placed within a delivery catheter. The guidewire may have multiple purposes, including but not limited to: guiding the delivery catheter and electrodes to a target site; providing electric potential to the distal tip (e.g., where the feeder electrodes may be positioned); providing field conformation; etc. The 3D configuration of the one or more feeder electrodes, combined with complementary return electrodes, guides the pattern of delivery of energy (e.g., for PFA). The voltage applied controls the field strength, while temporal variation in field strength may be used to modulate biologic effects.

Various electrode configurations may be used, including front firing, lateral firing (e.g., where the field has a component perpendicular to the delivery catheter/guidewire). The electrodes may also be positioned on flat opposing surfaces, which may or may not be parallel, including clamp configurations which can grasp tissue and apply an electric field. Additional electrode configurations which may be used include torus, spherical, ribbon and pyramidal configurations.

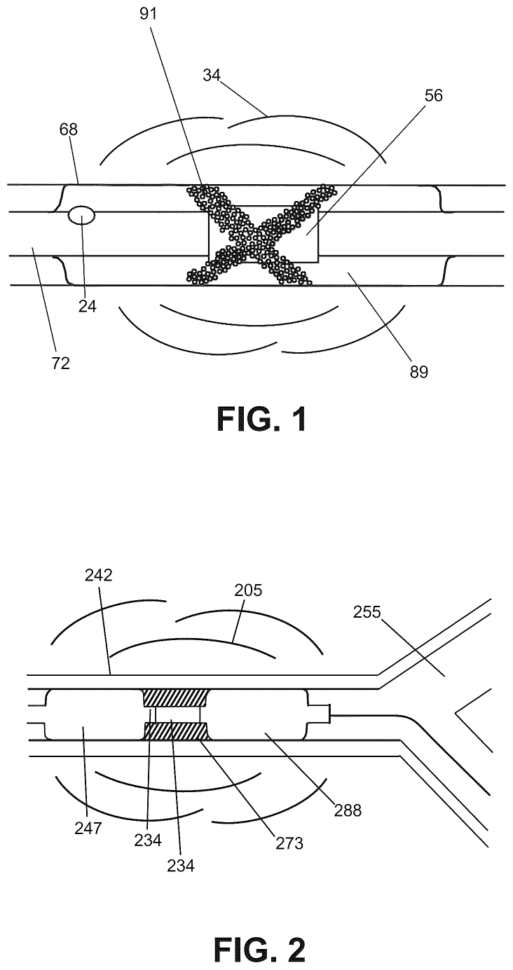

FIG. 1 shows aspects of a medical device that is inserted into a subject, the medical device including a catheter body 72 that is inserted through a vessel with vessel wall 68. The medical device here includes a port 24, from which a coupling medium 89 may be delivered towards an electrode 56 (e.g., a feeder electrode) that is positioned extending from the catheter body 72. The medical device also includes expandable elements in the form of a balloon with multiple perforations 91. Application of energy to the electrode 56 may be used to generate an electric field 34, the range of which is enhanced via the coupling medium 89 delivered to a target region in the vessel from the port 24 of the medical device.

FIG. 2 shows aspects of a medical device inserted into a subject, where the medical device includes multiple extendable elements including balloons 247 and 288. The balloons 247 and 288 are positioned near to target tissues 242 (e.g., nerves) in the subject which are to be treated. Port 234 is positioned between the balloons 247 and 288 of the medical device, and a coupling medium 273 is delivered therefrom. The coupling medium 273 surrounds electrode 265, thereby providing an extended virtual or fluid electrode that directly contacts the target tissues 242. FIG. 2 shows isoelectric lines 205 illustrating the effective electric field provided by the extended or fluid electrode (e.g., the combination of the electrode 265 and coupling medium 273. Here, the balloons 247 and 288 are positioned upstream of a bifurcation of vessel 255.

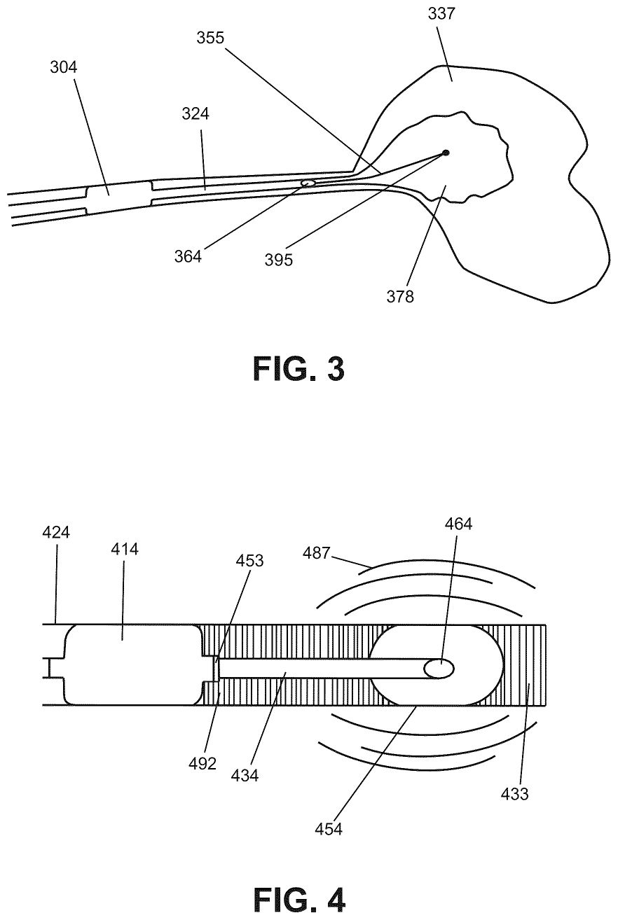

FIG. 3 shows a medical device inserted into a subject via a vessel near to a target tissue 337 to be treated (e.g., a tumor). Here, the medical device includes expandable elements in the form of a balloon 304 that is positioned on a catheter body 324. The catheter body 324 includes a port 364, from which non-conducting and coupling mediums may be delivered as desired. For example, the balloon 304 may be expanded to cut off blood supply to the target tissue 337, followed by delivery of a non-conducting medium from the port 364 to flush blood or other bodily fluids from the target tissue 337. A coupling medium may then be delivered via the port 364, and advanced toward the target tissue 337 proximate an end of a guidewire 355 that extends from the catheter body 324. One or more electrodes 395 (e.g., feeder electrodes) are positioned at a tip of the guidewire 355. The electrodes 395 and the coupling medium in contact therewith create a larger effective electrode volume 378 nearer to the target tissue than the catheter body 324 may be positioned. Coordination of the fluid delivery and electrical pulses provided from the electrode 395 allows for the boundary of the electrode volume 378 to advance through the target tissues 337, allowing for effective treatment thereof.

FIG. 4 shows a medical device inserted into a subject, including expandable element 414 (e.g., a balloon) that is expanded within a vessel 424. The expandable element 414 blocks blood flow downstream where an electrode 464 is positioned via a guidewire 434. A port 453 is included on the expandable element 414, from which a sequence of fluids is delivered. A non-conducting fluid 433 may be first delivered, followed by a bolus of conducting fluid 454 (e.g., a coupling medium), which is subsequently followed by additional non-conducting fluid 492. The initial non-conducting fluid 433 may flush the area of blood or other bodily fluids, and the additional non-conducting fluid 492 pushes the bolus of conducting fluid 454 forward until it reaches the electrode 464. The electrode 464 and the bolus of conducting fluid 454 provide a larger effective electrode area (e.g., than the electrode 464 alone). When energy is applied to the electrode 464, the larger effective electrode provides an electric field 487 as illustrated.

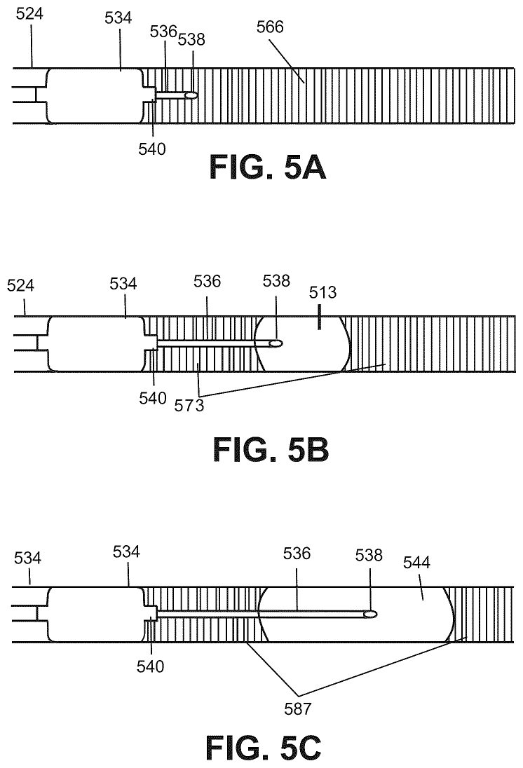

FIGS. 5a-5c show a medical device positioned within a vessel 524, the medical device including an expandable element 534 (e.g., a balloon) from which a guidewire 536 extends, with an electrode 538 being positioned at a tip of the guidewire 536. The medical device further includes port 540.

FIG. 5a shows a non-conducting fluid 566 that is delivered from the port 540, with the guidewire 536 being partially extended.

FIG. 5b shows a bolus of conducting fluid 513 positioned between non-conducting fluid 573 with the guidewire 536 being partially extended. This may be achieved through initial delivery of a portion of the non-conducting fluid 573, followed by the bolus of conducting fluid 513, followed by delivery of an additional portion of the non-conducting fluid 573 which pushes the bolus of conducting fluid 513 forwards towards the electrode 538 at the tip of the guidewire

FIG. 5c shows expansion of the conducting bolus as further coupling medium is introduced conducting fluid 544 is positioned between non-conducting fluid 587 with the guidewire 536 being further extended. Similar to FIG. 5B, this may be achieved through initial delivery of a portion of the non-conducting fluid 587, followed by the bolus of conducting fluid 544, followed by delivery of an additional portion of the non-conducting fluid 587 which pushes the bolus of conducting fluid 544 forwards towards the electrode 538 at the tip of the guidewire 536. Introduction of additional conducting fluid 513, 544 may be introduced from a port in the guidewire tip 538, while additional non-conducting fluid 587 may be introduced via a port 540 in the catheter. Coordinated introduction of fluid from either port may be performed to increase the bolus volume of the conducting fluid 513, 544 and/or to advance the conducting fluid bolus along the vessel by introducing additional non-conducting fluid 587 from the port 540. The electrode at the guidewire tip 538 provides a path into the conducting fluid 513, 544 to allow field generation in the surrounding tissues and fluids.

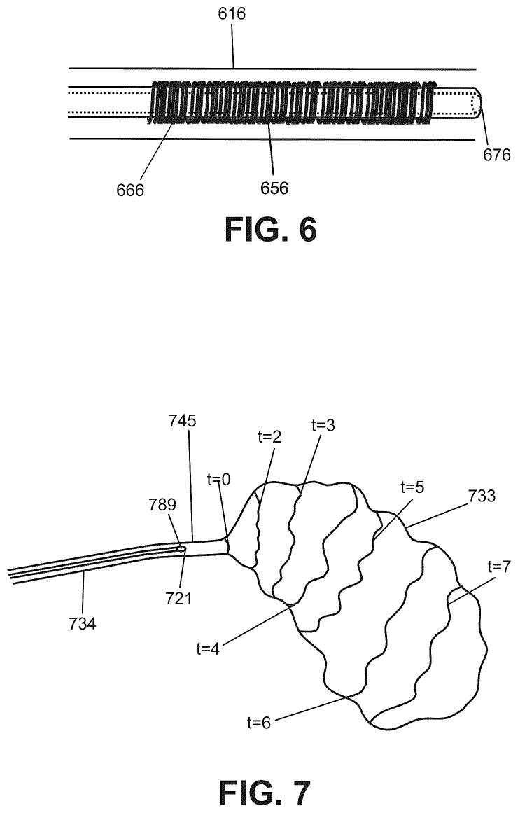

FIG. 6 shows aspects of a receiving or return electrode 656, which is positioned within a receiving vasculature 616 of a subject (e.g., to which a medical device with one or more feeder electrodes as described herein are inserted). The return electrode 656 advantageously includes a high surface area 666 for the return path (e.g., for PFA pulses delivered via one or more feeder electrodes of a medical device positioned proximate target tissues as described elsewhere herein). The structure may include a port 676 to deliver or retrieve fluid from the vessel. In some embodiments, the structure may be a guidewire, sized so as to fit into small vessels within the body. A section of the tip thereof may include a spring 666 in order to provide flexibility of the device, a region of the spring 666 may be exposed so as to provide an electrode function. The core of the guidewire may provide a fluid pathway with the tip exposing the core so as to provide a port 676 for providing a fluid delivery/retrieval function. In some embodiments, this simplified structure may allow for the diameter of the guidewire to be smaller than 1 mm, smaller than 0.5 mm, smaller than 0.25 mm, smaller than 0.15 mm, or the like.

FIG. 7 shows a medical device including an isolated guidewire body 734, which includes a port 721 and a feeder electrode 789 at a tip thereof. The isolated guidewire body 734 is positioned within a subject (e.g., one or more vessels thereof, not shown) proximate a target tissue 733 (e.g., a tumor). A coupling medium 745 is delivered via the port 721 as energy is applied to the feeder electrode 789 (e.g., to provide PFA pulses). As the coupling medium 745 is delivered from the port 721 towards the target tissue 733, moving waves of a maximal field gradient are illustrated at times t=0, t=1, . . . t=7.

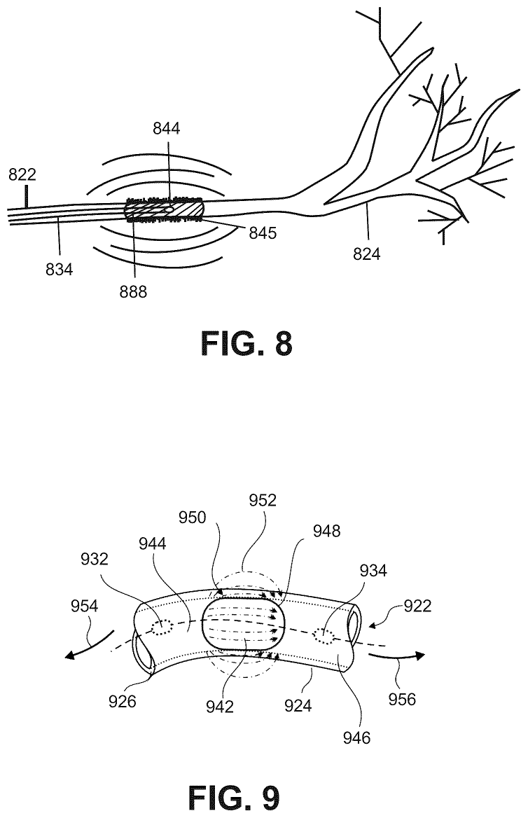

FIG. 8 shows a medical device including a guidewire 834 positioned within a vessel 822 (e.g., an artery, a vein, a lymphatic duct, etc.) upstream of its microvasculature 824. An electrode 844 is positioned at a tip of the guidewire 834. FIG. 8 shows a region of maximal field gradient 888 extending from a bolus of conducting fluid 845 positioned near to the electrode 844 at the tip of the guidewire 834. As the volume of the bolus of conducting fluid 845 increases and extends into the microvasculature 824, the region of maximal field gradient 888 will also expand into the microvasculature 824. The microvasculature 824 around the vessel 822 (e.g., an artery) provides blood to the nerves and other tissues around the vessel 822. Such microvasculature 824 structures will be penetrated by a coupling medium (e.g., the bolus of conducting fluid 845) as it advances therethrough, resulting in large electric fields within the microvasculature 824 during pulse application to the electrode 844.

FIG. 9 shows a medical device inserted into a vessel lumen 922 having a vessel wall 924, with microvasculature 926 in the vessel wall 924. The medical device includes respective electrodes 932, 934 which are placed upstream and downstream of a non-conducting bolus 942. The non-conducting bolus 942 is disposed between two conducting boluses 944 and 946. A meniscus 948 is formed between the non-conducting bolus 942 and the conducting boluses 944 and 946 as shown. A region with field concentration 950 is shown, along with field lines 952. The conducting boluses 944 and 946 may continue towards fluid controlled zones 954, 956. The meniscus 948 between the different fluids (e.g., the non-conducting bolus 942 and the conducting boluses 944 and 946). The non-conducting bolus 942 may include various alternative fluids, including but not limited to biodegradable polymers, fluorocarbon, hemoglobin-based oxygen carriers (HBOCs), perfluorocarbons (PFCs), lipid emulsions, polyoxyethylated castor oil, ethiodized oil (e.g., ethyl ester of the fatty acids of poppyseed oil), carbon dioxide gas bubbles (e.g., non-conducting, safe and easily absorbable), iodized poppyseed oil, human serum albumin, etc. The conducting boluses 944 and 946 may include various conducting fluids, including but not limited to strong ionic solutions, ionic conducting fluids, fluids including sclerosing agents, liquid metal fluids, ionic fluids with metallized microspheres (e.g., biocompatible materials like those used in stents, biodegradable metals and alloys, etc.), conjugated polymer fluids, biodegradable biocompatible liquid metals, etc.

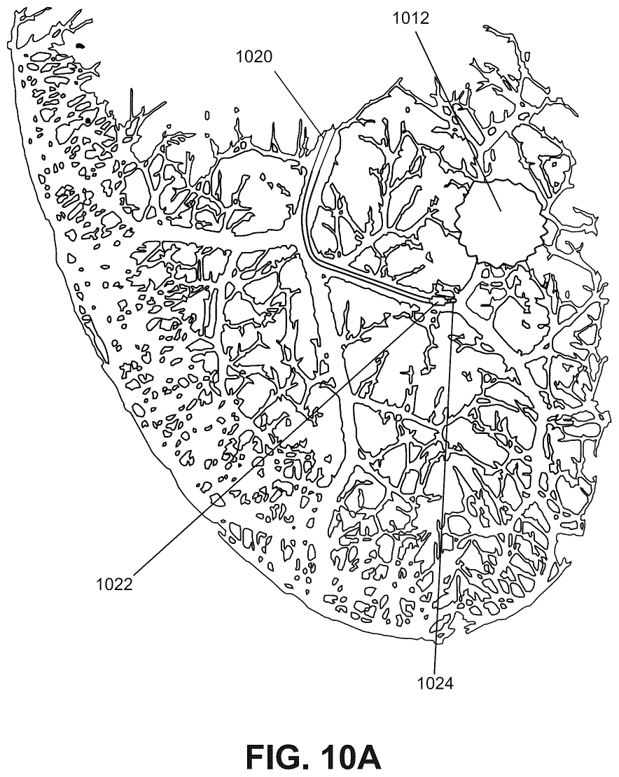

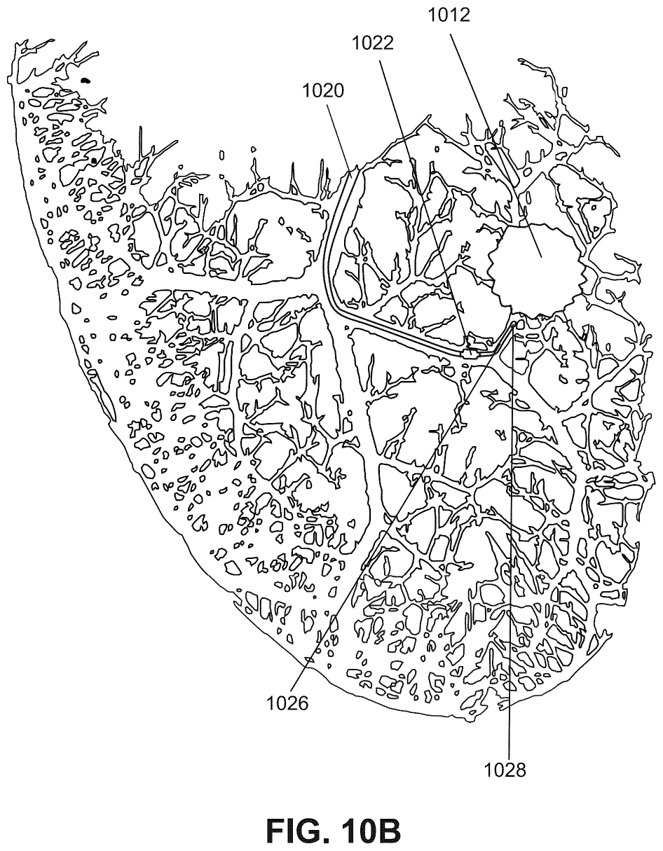

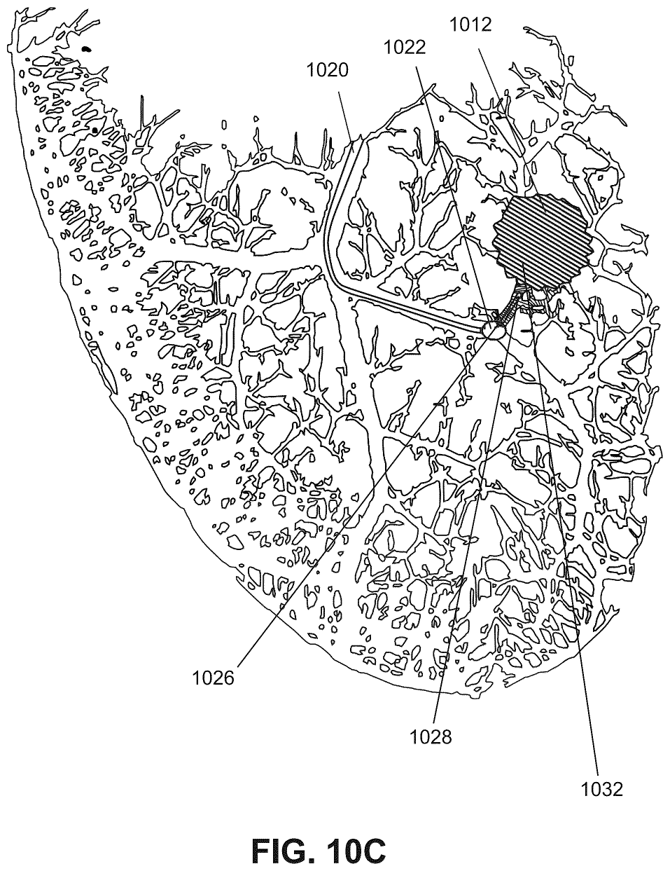

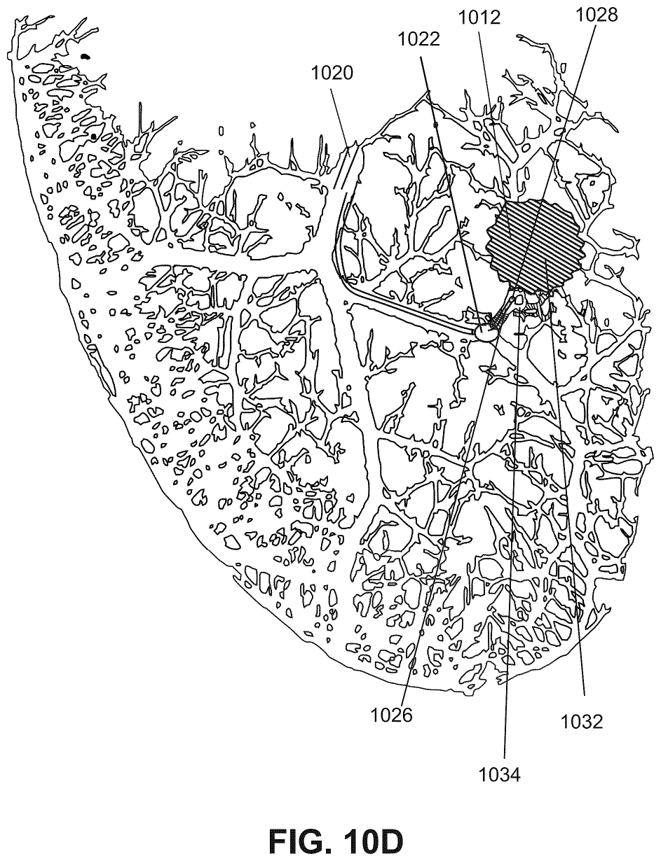

FIGS. 10a-10d show use of a medical device 1020 for treating a target region 1012 (e.g., microvasculature of a subject). The medical device 1020 shown in FIGS. 10a-10d is a balloon-based delivery catheter, including a balloon 1022 and a port 1024. The balloon 1022 of the delivery catheter medical device 1020 is advanced within the vasculature of the subject towards the target region 1012 (e.g., until reaching a spot where the balloon 1022 cannot fit. The balloon 1022 may be delivered to the area shown in FIG. 10a in a collapsed state, and may then be expanded to isolate the vasculature leading to the target region 1012 (e.g., to block blood flow thereto). As shown in FIG. 10b, a guidewire 1026 may then be advanced close to the target region 1012. The guidewire 1026 includes one or more ports and/or electrodes 1028 at a tip thereof. FIG. 10c shows fill with a non-conducting fluid 1032, which may be delivered via the ports/electrodes 1028 at the tip of the guidewire 1026. FIG. 10d shows introduction of a bolus of conducting fluid 1034 (e.g., a coupling medium), which may also be delivered via the ports/electrodes 1028 at the tip of the guidewire 1026.

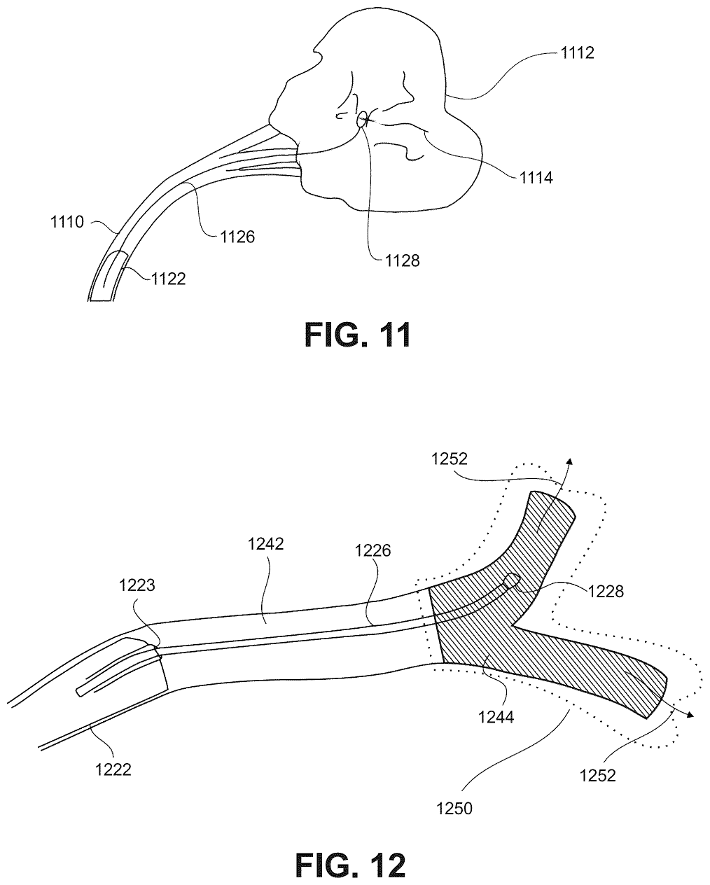

FIG. 11 shows a medical device including a balloon-based delivery catheter with a balloon 1122 positioned in a vessel 1110 proximate a target region 1112 to be treated (e.g., a tumor). Blood supply 1114 to the target region 1112 is also shown. A guidewire 1126 extends from the balloon 1122 towards the target region, where the guidewire 1126 includes an electrode 1128 (e.g., a feeder electrode) at a tip thereof.

FIG. 12 shows a catheter-based medical device including one or more expandable elements 1222 at a tip 1223 of the catheter, positioned within a vessel 1210. A guidewire 1226 extends from the catheter tip 1223, with an electrode 1228 being positioned at a tip of the guidewire 1226. FIG. 12 also shows a non-conductive fluid 1242 and a conductive fluid 1244 which may be delivered from one or more ports on the guidewire 1226 and/or the expandable elements 1222. Shown in the FIG. 12 is the expansion 1250 of the therapeutically effective region around the advancing fluid electrode, the field gradients accentuated by the natural anatomical features of the anatomy as the fluid is advanced 1252 therethrough.

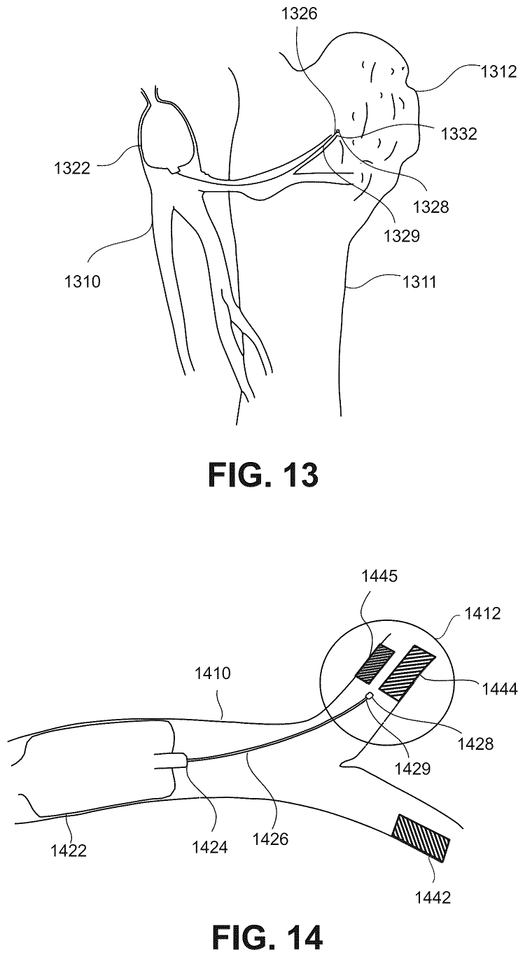

FIG. 13 shows a balloon-based catheter medical device including a balloon 1322 positioned within a vessel 1310 (e.g., deep femoral artery branches proximate a femur bone 1311). A guidewire 1326 extends from the balloon 1322 towards a target region 1312 (e.g., a tumor). The guidewire 1326 includes an electrode 1328 at a tip thereof (e.g., a feeder electrode) as well as a port 1329 along a length thereof (e.g., from which non-conducting and coupling mediums may be delivered as described elsewhere herein). FIG. 13 also shows a fluid migration vector 1332 for non-conducting and coupling mediums delivered from the port 1329 of the guidewire 1326.

In some embodiments, the approach may be used to access the marrow of the bone, and the ablation applied so as to defunctionalize and/or sterilize the marrow, targeting carcinogenic cells or viral particles that may take up space therein.

FIG. 14 shows a balloon-based catheter medical device includes a balloon 1422 positioned within a vessel 1410 upstream of a bifurcation, with a target region 1412 along one of the vessel branches downstream of the bifurcation as illustrated. A guidewire 1426 extends from a port 1424 at a tip of the balloon 1422, with the guidewire 1426 having one or more electrodes 1428 and ports 1429 proximate a tip thereof. FIG. 14 also shows a non-conductive fluid 1442 and a coupling medium 1444 which may be delivered via the ports 1424, 1429, along with isometric lines 1445 representing pulses delivered via the electrode 1428.

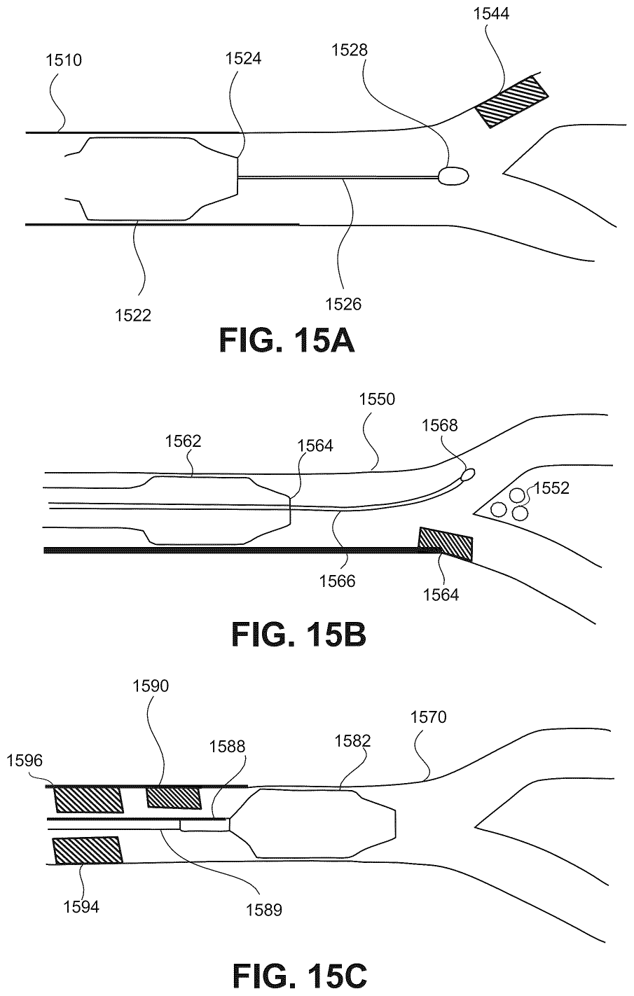

FIGS. 15a-15c shows aspects of medical devices positioned in branching vessels 1510, 1550, 1580. FIG. 15a shows a medical device with an expandable element 1522 positioned in branched vessel 1510, a port 1524 and an extendable element 1526 that extends from the port 1524 on the expandable element 1522. An electrode is positioned at a tip of the extendable element 1526. A coupling medium 1544 is also shown delivered along one of the branches of the branched vessel 1510.

FIG. 15b shows a medical device including a balloon-based delivery catheter having a balloon 1562 positioned in branched vessel 1550. The delivery catheter includes one or more ports 1564 on the balloon 1562, along with a guidewire 1566 with an electrode 1568 at a tip thereof that extends towards one of the branches of the branched vessel 1510 proximate a target region 1552 (e.g., target ganglia). A coupling medium 1564 is also shown delivered near the branch of the branched vessel near to the target region 1552.

FIG. 15c shows a medical device including a balloon-based delivery catheter having a balloon 1582 positioned in branched vessel 1570. An electrode 1588 is shown extending from a surface of the balloon 1582, along with a port 1589 from which various fluids may be delivered as shown, including an electric fluid 1590 and a coupling medium 1594. Also shown is blood 1596.

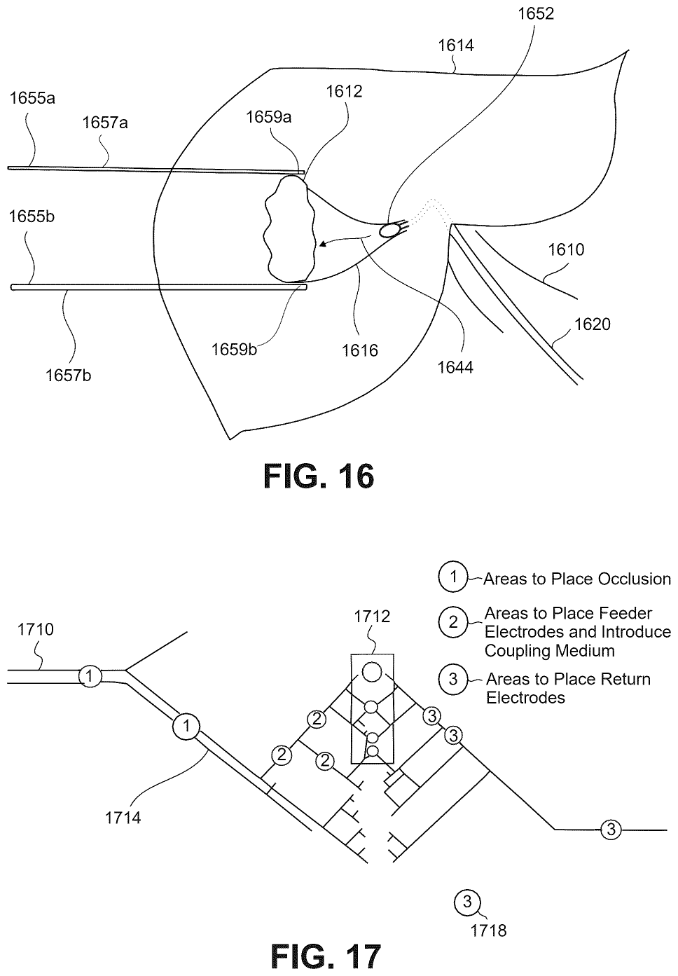

FIG. 16 shows an example of combined percutaneous and vascular approaches for therapy, in which a catheter-based medical device 1620 is positioned in a vessel 1610 proximate a target region 1612 (e.g., a tumor), which in this example is part of a liver 1614 of a subject. A fluid fill direction 1644 is also shown for non-conducting fluids and coupling mediums delivered via the catheter-based medical device 1620 towards an arterial supply 1616 of the target region 1612. An occlusive element 1652 is also shown, which aids in stopping or containing fluid flow through the target region 1612. Here, in addition to use of the catheter-based medical device 1620 for delivery of non-conducting and coupling mediums near to the target region, one or more needle electrodes 1655a, 1655b are inserted proximate the target region 1612. Each of the needle electrodes 1655a, 1655b may include an insulated region 1657a, 1657b and an exposed electrode tip 1659a, 1659b. Electrical pulses may be delivered via between the needle electrodes 1655a. 1655b (e.g., for PFA), where non-conductive and coupling mediums delivered via the catheter-based medical device 1620 may be used to improve the effective electrode area in a manner similar to that described elsewhere herein with respect to feeder electrodes that are part of catheter-based medical devices.

In some embodiments, a non-conducting medium is passed through target tissues to displace any conducting biological fluids from the vicinity of the target tissues. This provides various advantages, including in allowing for more uniform field generation in the target tissues, and allowing for lower energy to be applied (e.g., via the needle electrodes 1655a, 1655b) in order to reach therapeutic field levels in the target tissues. Conducting mediums may also be used in some embodiments, such as to maximize current flow through the target tissues (e.g., during thermal ablation procedures) while minimizing stray current flow elsewhere. The non-conducting medium may be used in some embodiments for maximizing field levels (e.g., for PFA), and conducting medium may be used for maximizing current flow (e.g., for thermal ablation methods).

FIG. 17 illustrates therapies for a target tissue 1712 accessible via an artery 1710 and associated nerves 1714 (e.g., which can be treated anywhere along the tree of nerves shown). FIG. 17 also shows various areas in and around the target tissue 1712, including: areas labeled “1” where occlusion elements may be placed (e.g., expandable elements, such as balloons, which may block blood flow to the target tissue 1712); areas labeled “2” where feeder electrodes may be placed and coupling media may be introduced (e.g., via ports on a medical device, which may be proximate the occlusion elements and/or feeder electrodes); and areas labeled “3” where receiving or return electrodes may be placed, including a remote region 1718 placed elsewhere on the body (e.g., a patch electrode on skin of a subject).

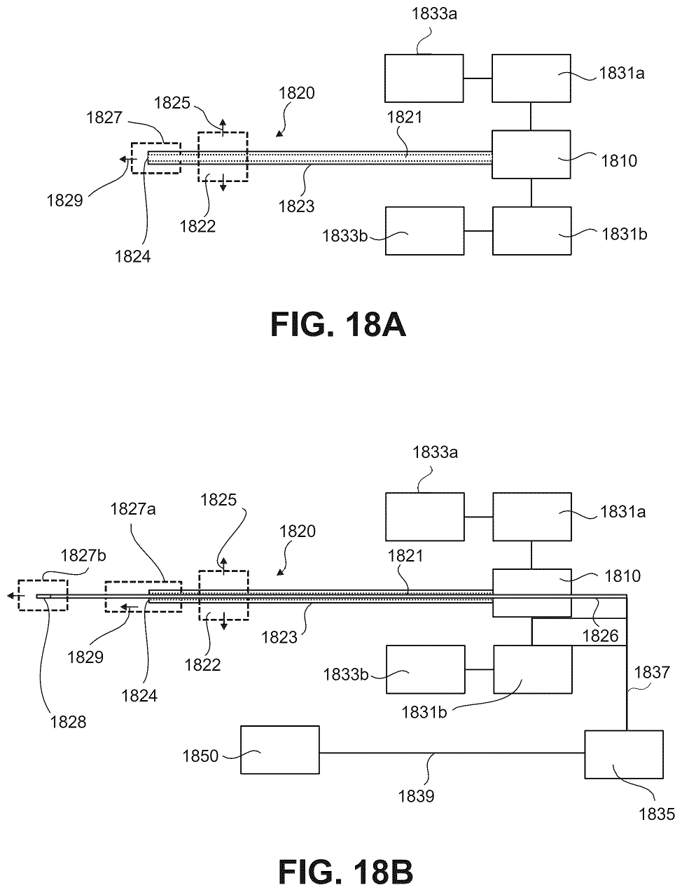

FIG. 18a shows a medical device 1820 (e.g., a catheter-based medical device) which includes one or more lumens 1821 which are coupled to a handle 1810 and contained within catheter walls 1823. The lumens 1821 may include a catheter body, one or more guidewires (e.g., guidewire 1826 shown in FIG. 18b) configured to extend from a tip of the catheter body, etc. The medical device 1820 further includes an expandable element 1822 configured to expand 1825, and one or more ports 1824 (e.g., at a tip of the catheter body) providing a fluid delivery zone 1827. The fluid delivery zone 1827 may advance as shown via element 1829 representing fluid boundary movement. The handle 1805 is coupled to reservoirs 1831a, 1831b associated with respective controllers 1833a, 1833b. The reservoir 1831a may provide a reservoir for delivery fluids (e.g., non-conducting and coupling media) which are deliverable along one of the lumens 1821 via the port 1824. The controller 1833a may provide an infusion pump or other means for controlling such fluid delivery from the reservoir 1831a. The reservoir 1831b may provide a reservoir for fluid used to expand the expandable element 1822 (e.g., fluid used to fill a balloon and isolate regions downstream from the expanded 1825 expandable element 1822). The controller 1833b may provide an infusion pump or other means for controlling such fluid delivery from reservoir 1831b.

FIG. 18b shows additional aspects of the medical device 1820, including a guidewire 1826 that extends through the lumens 1821 and has a feeder electrode 1828 at a tip thereof. The feeder electrode 1828 is coupled via the guidewire to a generator 1835 providing an energy delivery path 1837 for the feeder electrode 1828. The generator 1835 is also coupled to a return electrode 1850 on a return path 1839, where the return electrode may be in a vessel, on the body of a subject, on the catheter walls 1823 some distance away from the feeder electrode 1828, in a vein, etc. Whereas FIG. 18a shows a single fluid delivery zone 1829, FIG. 18b shows two distinct fluid delivery zones 1829a and 1829b, the first fluid delivery zone 1829a at an end of the lumens 1821 of the catheter body and the second fluid delivery zone 1829b surrounding the feeder electrode 1828. The first fluid delivery zone 1829a may have non-conducting fluid delivered thereto, while the second fluid delivery zone 1829b may have a coupling medium delivered thereto.

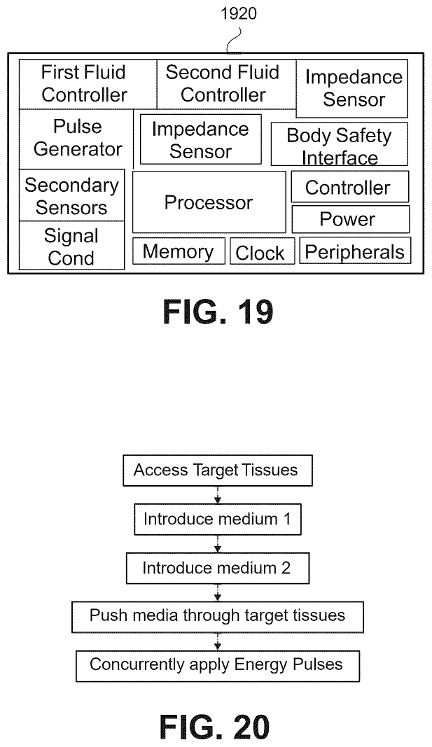

FIG. 19 shows a hardware block diagram of a medical device 1920, which includes a processor, memory, clock, peripherals, signal conditioning circuitry, power, a controller, a body safety interface, an impedance sensor, one or more secondary sensors, first and second fluid controllers (e.g., for delivering non-conducting and coupling media), and a pulse generator (e.g., for applying pulses or energy to feeder electrodes).

The medical devices described herein may be used for providing therapy to various target regions, including various organs, tissues, nerves, tumors, ganglion sites, etc. Therapy may include innervation along a target anatomy, innervation within the organ parenchyma, smooth muscle innervation in arteries, fluid transfer into the microvasculature around vessels, targeting organ resurfacing, ganglion access which may be combined with recordings for ganglia localization. Internal vessel and external approaches are enabled. The medical devices described herein may be used with methods for determining when a procedure or therapy is completed, for determining when fluids are in the right place to start a procedure, for infusing multiple fluids in sequence to create desired gaps therebetween and to create field support volumes, for sucking up fluids from a medical device in the veins after a procedure or therapy is completed, for providing bipolar asymmetrically undulating PFA pulses, etc.

FIG. 20 shows a method which may be performed using the medical devices described herein. The method begins with accessing target tissues, such as by delivering a catheter via one or more vessels of a subject to access a region proximate the target tissues. This may include placing the catheter, expanding one or more expandable elements thereof, and then delivering a guidewire or other extendable element further towards the target tissues. The method then continues with introduction of a first medium (e.g., a non-conducting medium) followed by a second medium (e.g., a coupling medium) via one or more ports of the medical device (e.g., which may be on or near the expandable elements and/or extendable elements of the medical device). The first medium flushes blood or other bodily fluids from the target tissues. The method then continues with pushing the second medium through the target tissues (e.g., which may be achieved through introducing more quantity of the second medium and/or the first medium). As the second medium is pushed through the target tissues, energy pulses are concurrently applied (e.g., via one or more feeder electrodes of the medical device, via external needle electrodes, etc.).

In some embodiments, the first and second media comprise respective non-conductive and conductive media, which are introduced in a sequence of non-conductive→conductive→non-conductive, with the sequence of introduced media pushing the conductive media through the anatomy until it contacts feeder electrodes of the medical device, and then energy pulses may be applied. In some embodiments, the feeder electrodes are placed in an artery and return electrodes are placed in a vein. The return electrodes may also or alternatively be placed elsewhere (e.g., a different location in the artery possibly on the delivery catheter, on a body patch applied to a skin of the subject, etc.). The electric field fans out from the boundary of the conductive medium. The non-conductive medium may by pushed through the target tissues, followed by the conductive medium from within the volume of the non-conductive medium, with additional non-conductive medium being introduced upstream to push the conductive medium through the target tissues while pulsing energy to the feeder electrodes.

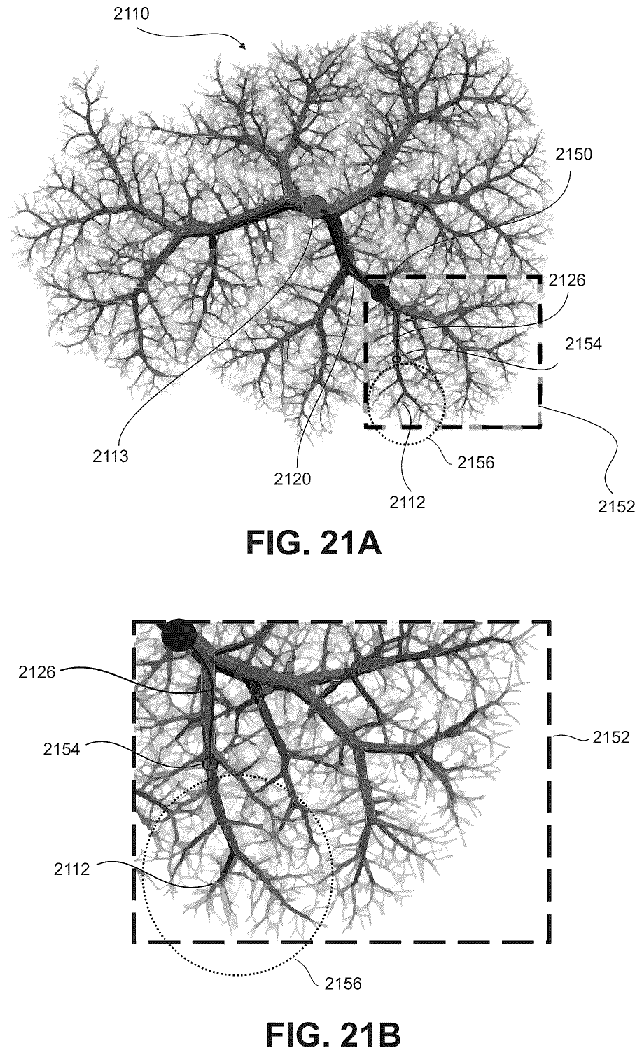

FIGS. 21a and 21b illustrate a medical device which is inserted into an arterial supply of a region of an organ 2110, which is used for accessing target tissues 2112 therein. A main lobe artery entry point 2113 for the medical device is shown, where a catheter body 2120 is inserted. The catheter body 2120 is inserted via the main lobe artery point 2113 and is advanced to a first site 2150. The first site 2150 represents a first fluid control and introduction site. A non-conducting medium may be introduced at the first site 2150 (e.g., from one or more ports on the catheter body 2120 or expandable elements thereof), and extends throughout a first fluid introduction region 2152. A guidewire 2126 extends from the catheter body 2120 extending from the first site towards a second site 2154. The second site 2154 represents a location of one or more feeder electrodes on the guidewire 2126. The second site 2154 may also include one or more ports on the guidewire from which additional fluid is introduced (e.g., a coupling medium). The fluid introduced at the second site 2154 extends through fluid introduction region 2156. FIG. 21b shows a close-up view of the region 2152.

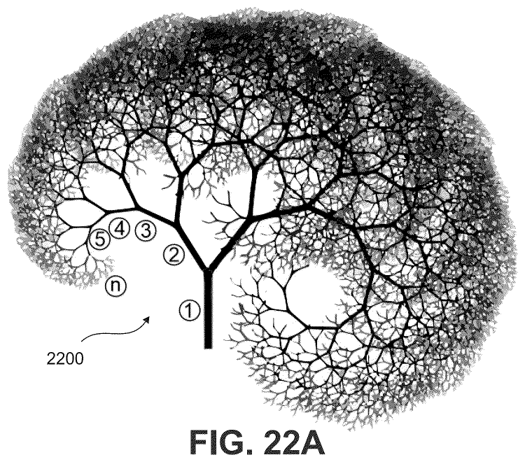

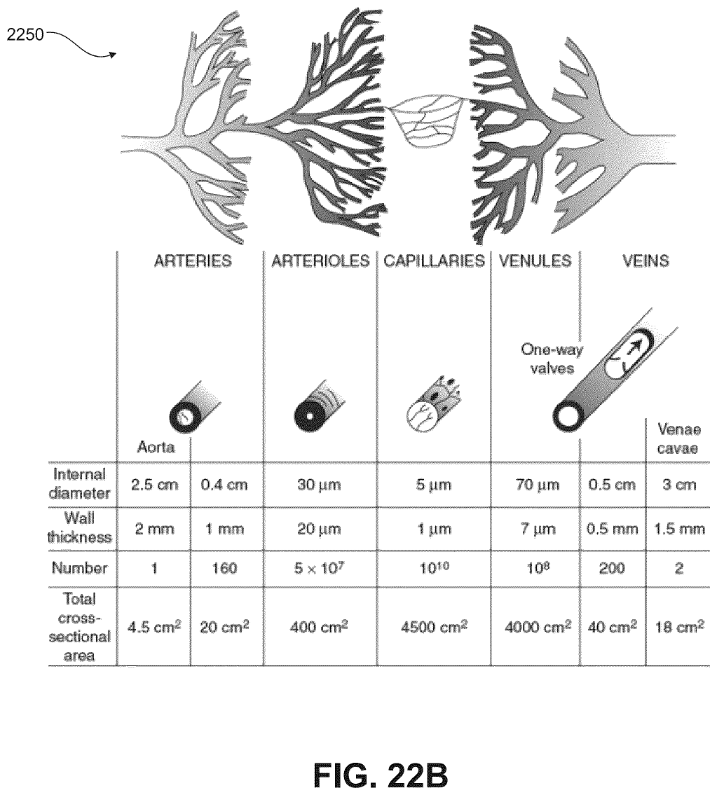

FIG. 22a shows a fractal tree structure 2200, which may represent a portion of microvasculature a subject that includes one or more target tissues to be treated. For illustration, the numbers 1, 2, . . . n indicate branch stages. With increasing branches, the cross sectional area increases up to the capillaries and so the length of an introduced bolus decreases and overall flow velocity slows. Electric fields across the bolus grow in vessels with increasing n accordingly. With an increasing branch number, the vessels transition from arteries, to arterioles, to capillaries, with the overall cross section increasing accordingly. Some example dimensions are illustrated in the table 2250 of FIG. 22b.

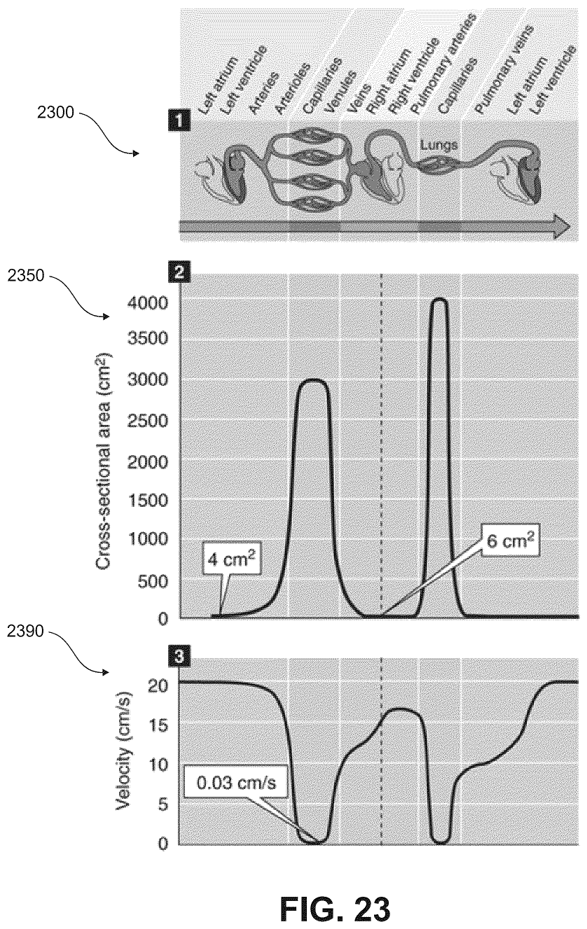

FIG. 23 shows an illustration 2300 of different parts of an anatomy of a subject, illustrating a sequence of blood flow through the left atrium, left ventricles, arteries, arterioles, capillaries, venules, veins, right atrium, right ventricle, pulmonary arteries, capillaries, pulmonary veins, left atrium and left ventricle. FIG. 23 also shows a plot 2350 of cross-sectional area of the different parts of the anatomy of the subject, and a plot 2390 of velocity (e.g., of a bolus of fluid) through the different parts of the anatomy of the subject.

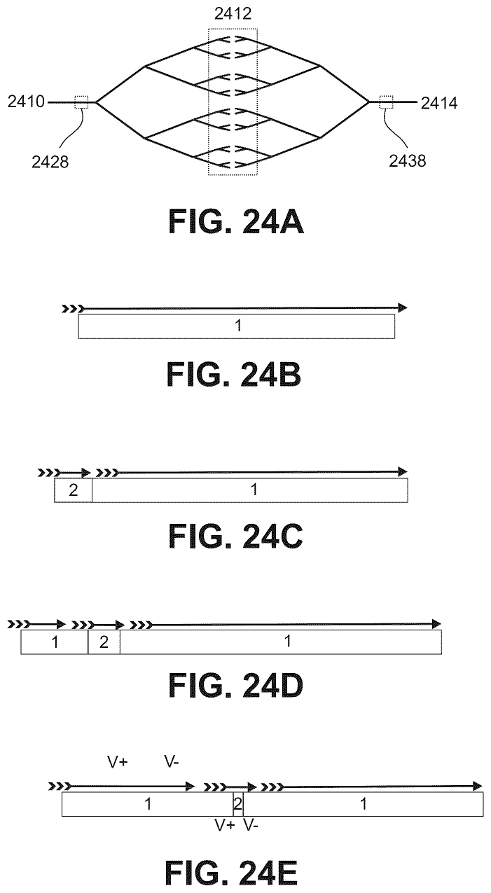

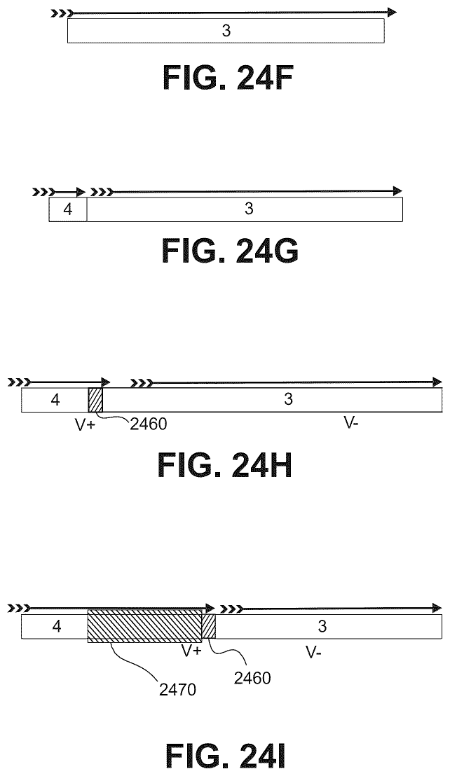

FIGS. 24a-24i show aspects of fluid flow of coupling and non-coupling (e.g., insulating or non-conducting) media. FIGS. 24a shows a feeder electrode 2428 of a medical device placed in an artery 2410 feeding target tissues 2412, with a return electrode 2438 placed in a vein 2414 also connected to the target tissues 2412. FIGS. 24b-24e show a first method for therapy, where “1” represents a coupling fluid or medium, while “2” represents a non-conducting/insulating fluid or medium. As shown in FIG. 24b, initially the coupling fluid is delivered to touch both the feeder electrode 2428 and return electrode 2438. Next, the insulating fluid is introduced as shown in FIG. 24c near to the feeder electrode 2428. More coupling fluid is then introduced as shown in FIG. 24d. More coupling fluid continues to be introduced as shown in FIG. 24e, where the distance between the first and second portions of the coupling fluid (e.g., separated by the insulating fluid) is reduced as the fluids enter the region with the highest cross-sectional area. As the fluids are passed through the target tissues 2412, pulses are applied between the feeder electrode 2428 and the return electrode 2438, where an electric field resulting from such pulses is maximized as the fluids pass through the capillaries (e.g., enabling performance of ablation with a reasonably low voltage). The approach may allow for maximization of electrical field in the smallest vessels of the target tissues while minimizing electrical field in and around the larger vessels near to the target tissues, thus providing an extra degree of protection to tissues outside of the target tissues and therapeutic selectivity of the target tissues during a procedure. A substantially lower ablation potential applied between the electrodes may reduce the far field risk of muscle contractions during the application of therapy to the target tissues.

In some embodiments, a method includes identifying the smallest arteries feeding target tissues 2412, and blocking blood flow through the target tissues 2412. A first electrode (e.g., the feeder electrode 2428) is positioned in the smallest artery 2410 near to the target tissues 2412, and a second electrode (e.g., the return electrode 2438) is positioned in the receiving veins 2414. The region between the feeder electrode 2428 and the return electrode 2438 is filled with the coupling medium (e.g., conductive fluid 1) as illustrated in FIG. 24b. A bolus of the non-conducting medium (e.g., insulating fluid 2) is filled into the artery 2410, with the bolus volume being filled such that the first fluid length is equal to “x” (e.g., 1 mm, 5 mm, 10 mm, etc.) as illustrated in FIG. 24c. Additional coupling medium is introduced to push the fluid through the target tissues 2412, as is illustrated in FIGS. 24d and 24e. The effective length of the coupling medium in the vessels changes inversely proportional to the cross-sectional area of the vessels (e.g., the effective fluid length may decrease ⅕th, 1/10th, 1/50th, etc. of the original length as it passes through the smallest arterioles and capillaries). On either side of the bolus, the coupling medium are in electrical connection with the feeder electrode 2428 and the return electrode 2438. Electrical pulses are produced between the feeder electrode 2428 and the return electrode 2438 so as to produce large electrical fields in the region of the bolus of the non-conducting medium (e.g., the bolus of insulating fluid 2). In this example, the target electrical field in the smallest arterioles and capillaries of the target tissues may be set for roughly 500 V/cm, 1,000 V/cm, 1,500 V/cm, 2,000 V/cm. Given the change in length of the bolus of the non-conducting medium as it passes through the tiny vessels in the target tissues 2412, a suitable pulse voltage of less than 20V, less than 50V, less than 100V, less than 250V, etc. may be sufficient in order to create the target electrical fields in the adjacent tissues. The coupling medium is advanced until the feeder electrode 2428 and return electrode 2438 are once again in electrical communication with each other (e.g., the bolus of the non-conducting medium has passed completely between the feeder electrode 2428 and the return electrode 2438).

FIGS. 24f-24i illustrate a second method of therapy which may be performed using the medical device with the feeder electrode 2428 placed in the artery 2410 feeding the target tissues 2412 and the return electrode 2438 placed in the vein 2414 as shown in FIG. 24a. In FIGS. 24f-24i. “3” represents a non-conducting/insulating fluid or medium, while “4” represents a conducting/coupling fluid or medium. As shown in FIG. 24f, initially the non-conducting medium is introduced to displace blood from the target tissues 2412. Next, a bolus of the coupling medium is introduced near to the feeder electrode 2428 as shown in FIG. 24g. More of the coupling medium is introduced as shown in FIG. 24h, with the region of maximum field 2460 shown. As shown in FIG. 24i, the coupling medium continues to be introduced so as to advance it through the target tissues 2412, resulting in a treated region 2470 as the region of maximum field 2460 progresses along towards and through the target tissues 2412.

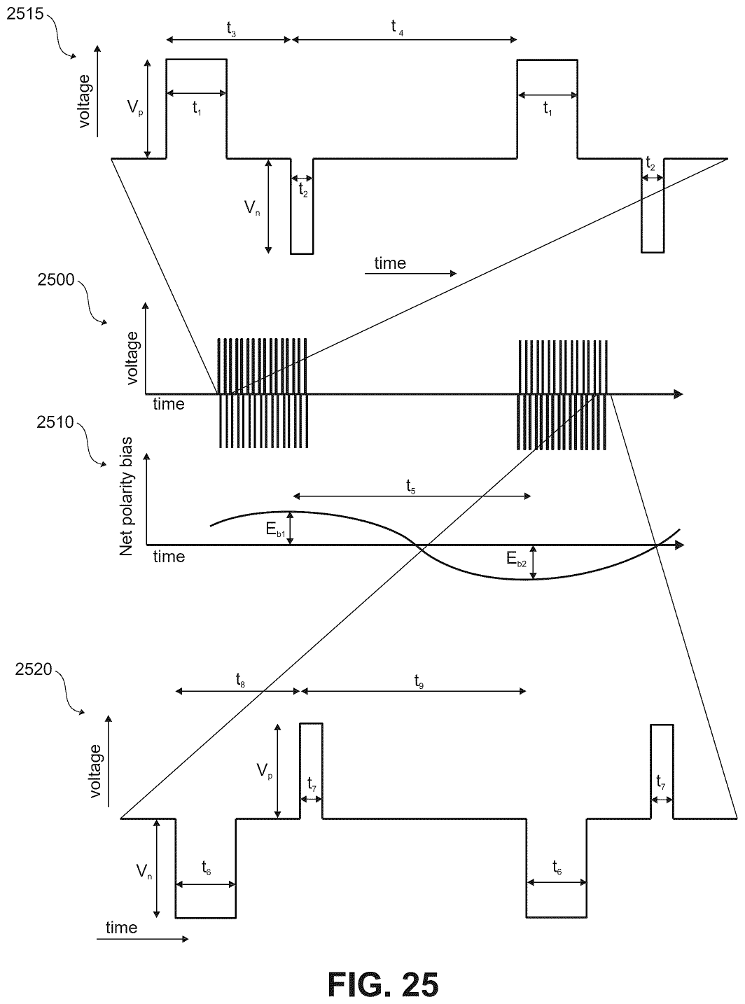

FIG. 25 shows plots 2500 and 2510 illustrating a pulse train including asymmetric pulses of opposite polarity. The plot 2500 illustrates applied voltage over time for a pulse train, while plot 2510 illustrates a net polarity bias of the pulse train over time. Plots 2515 and 2520 show detailed views of portions of the pulse train in the plot 2500, where plot 2515 shows two sets of asymmetric pulses that have longer positive voltage pulses and plot 2520 shows two sets of asymmetric pulses that have longer negative voltage pulses. In the plot 2515, positive voltage pulses with amplitude Vₚ are applied for a time t₁, with each of the positive voltage pulses being followed by a negative voltage pulse with amplitude Vₙ that is applied for a time t₂ (e.g., where t₂<t₁). The negative voltage pulses are applied at a time t₃ from a beginning of time t₁. The time between the sets of asymmetric pulses is denoted time to. As illustrated in plot 2510, the pulse train includes a changing polarity bias from an amplitude of E_{b1} (e.g., +100%) through to an amplitude of E_{b2} (e.g., −100%) over a time period denoted t₅. In the plot 2520, negative voltage pulses with amplitude Vₙ are applied for a time t₆, with each of the negative voltage pulses being followed by a positive voltage pulse with amplitude Vₚ that is applied for a time t₇ (e.g., where t₇<t₆). The positive voltage pulses are applied at a time t₈ from a beginning of time t₆. The time between the sets of asymmetric pulses is denoted time t₉.

Overall signal bias may be adjusted by altering the ratio between the positive pulse voltage and negative pulse voltage, by adjusting the ratio between the positive pulse width and negative pulse width, or the like. Such alterations may be completed at a frequency with which the bias shifts from positive to negative and back, and may be on the order of greater than 1 kHz, greater than 10 kHz, greater than 100 kHz, or the like. The shifting asymmetry throughout the pulse train allows for the application of asymmetric pulses to the tissue (e.g., thus potentially lowering the ablation thresholds thereof), while providing short-term charge asymmetry to the target tissues, maintaining a long-term neutral overall energy delivery and minimizing long-term charge imbalance around the treatment site.

In some embodiments, one or more sensors may be applied to the body of the subject, the sensors configured to monitor for changes in local charge accumulation and/or electric field during the application of pulses to the subject. The bias of the pulse train may be adjusted to prevent the long-range charge accumulation and/or potential from increasing beyond a threshold, such as a threshold needed to stimulate muscles, muscle endplates, and/or nerves in the far field regions of the body of the subject.

In some embodiments, where the local electric field is to be extended beyond a therapeutic threshold in local tissues for a period, the applied pulses may be substantially square wave in nature.

In some embodiments, the shape and frequency content of the waveform may be adjusted to selectively target tissue types within the target tissues (e.g., stem cells, bone cells, blood cells, muscle cells, fat cells, skin cells, nerve cells, endothelial cells, sex cells, pancreatic cells, and/or cancer cells).

In some embodiments, in applications where heating is considered in conjunction with field generation, a more sinusoidal waveform may be applied to the tissues.

In some embodiments, a medical device comprises an elongate member, one or more expandable elements positioned on the elongate member, one or more ports, one or more electrodes, and a controller. The controller is configured to actuate the one or more expandable elements within a vessel of a subject to block fluid flow through the vessel downstream to a target tissue site, to deliver a bolus of conducting medium via the one or more ports towards the target tissue site, and to apply energy pulses via the one or more electrodes for therapeutic electroporation while the bolus of the conducting medium is in contact with the one or more electrodes and extends from the one or more electrodes towards the target tissue site.

The elongate member may comprise a delivery catheter. The one or more expandable elements may comprise one or more balloons positioned on the delivery catheter.

The medical device may further comprise one or more extendable elements, the controller being further configured to advance the one or more extendable elements past a tip of the elongate member from a first site in the vessel to a second site close to the target tissue site than the first site. The one or more extendable elements may comprise a guidewire.

At least one of the one or more ports may be positioned on at least one of the one or more extendable elements. Said at least one of the one or more ports may be positioned proximate a tip of said at least one of the one or more extendable elements.

At least one of the one or more electrodes may be positioned on at least one of the one or more extendable elements. Said at least one of the one or more electrodes may be positioned proximate a tip of said at least one of the one or more extendable elements.

At least one of the one or more electrodes and at least one of the one or more ports may be positioned on at least one of the one or more extendable elements. Said at least one of the one or more electrodes and said at least one of the one or more ports may be positioned proximate a tip of said at least one of the one or more extendable elements.

The medical device may further comprise one or more fluid reservoirs containing the conducting medium.

The controller may be further configured to deliver a bolus of non-conducting medium prior to delivery of the bolus of conducting medium, the bolus of non-conducting medium flushing one or more bodily fluids from the target tissue site. The medical device may further comprise two or more fluid reservoirs, a first one of the two or more fluid reservoirs containing the conducting medium and a second one of the two or more fluid reservoirs containing the non-conducting medium.

The medical device may further comprise one or more infusion pumps, wherein the controller utilizes the one or more infusion pumps to deliver the bolus of the conducting media via the one or more ports towards the target tissue site.

The medical device may further comprise one or more generators, wherein the controller utilizes the generators to apply the energy pulses via the one or more electrodes for the therapeutic electroporation while the bolus of the conducting medium extends from the one or more electrodes towards the target tissue site.

The one or more electrodes may comprise one or more feeder electrodes and one or more return electrodes, the energy pulses being applied between respective pairs the one or more feeder electrodes and the one or more return electrodes. The one or more return electrodes may be positioned on the elongate member remote from the one or more feeder electrodes. The one or more return electrodes may also or alternatively be positioned on an additional elongate member positioned within another vessel. The controller is configured to measure impedance between the one or more feeder electrodes and the one or more return electrodes to determine when the bolus of conducting medium is in contact with the one or more feeder electrodes.

In some embodiments, a system comprises a first medical device comprising an elongate member, one or more expandable elements positioned on the elongate member, and one or more ports, a second medical device comprising one or more needle electrodes, and at least one controller. The at least one controller is configured to actuate the one or more expandable elements of the first medical device within a vessel of a subject to block fluid flow through the vessel downstream to a target tissue site, to advance the one or more needle electrodes of the second medical device into the target tissue site, to deliver a bolus of non-conducting medium via the one or more ports of the first medical device towards one or more needle electrodes of the second medical device inserted into the target tissue site to displace one or more biological fluids in a vicinity of the target tissue site, and to apply energy pulses via the one or more needle electrodes of the second medical device for therapeutic electroporation.

At least one of the one or more needle electrodes may comprise an insulated region along a length thereof and exposed electrode region at a tip thereof.

The at least one controller may comprise a first controller comprised within the first medical device, the first controller being configured to actuate the one or more expandable elements of the first medical device and to deliver the bolus of non-conducting medium, and a second controller comprising within the second medical device, the second controller being configured to advance the one or more needle electrodes and to apply the energy pulses.

The one or more needle electrodes may comprise at least a first needle electrode and a second needle electrode, and the at least one controller may be further configured to measure an impedance between the first and second needle electrodes to determine when the bolus of the non-conducting medium is in contact with the one or more needle electrodes.

In some embodiments, a method comprises delivering an elongate member of a medical device to a first site within a vessel proximate a target tissue site to be treated and actuating one or more expandable elements of the medical device to block flow of one or more bodily fluids through the vessel downstream from the first site to the target tissue site. The method also comprises advancing, via one or more ports of the medical device, a bolus of conducting medium, the bolus of conducting medium extending from one or more electrodes of the medical device, positioned at a second site, towards the target tissue site. The method further comprises applying, via one or more electrodes of the medical device, energy pulses to the target tissue site, the bolus of conducting medium extending a field gradient of the energy pulses from the second site to the target tissue site.

The second site may comprise a tip of one or more extendable elements of the medical device. The method may further comprise, prior to advancing the bolus of the conducting medium, advancing a non-conducting medium via the one or more ports of the medical device through the target tissue site to flush the one or more bodily fluids from the target tissue site.

Advancing the bolus of the conducting medium may comprise introducing the bolus of the conducting medium via the one or more ports of the medical device and introducing additional non-conducting medium via the one or more ports of the medical device to advance the bolus of the conducting medium from the second site towards the target tissue site.

Advancing the bolus of the conducting medium may comprise advancing a first bolus of the conducting medium through the target tissue site to extend from at least a first one of the one or more electrodes providing a feeder electrode for the one or more energy pulses to at least a second one of the one or more electrodes providing a return electrode for the one or more energy pulses, advancing a bolus of non-conducting medium past the feeder electrode, and advancing a second bolus of the conducting medium to a region surrounding the feeder electrode, wherein the one or more energy pulses are applied while the first bolus of the conducting medium is in a region surrounding the return electrode and while the second bolus of the conducting medium is in the region surrounding the feeder electrode.

Advancing the bolus of the conducting medium may comprise advancing a first bolus of non-conducting medium through the target tissue site to extend from at least a first one of the one or more electrodes providing a feeder electrode for the one or more energy pulses to at least a second one of the one or more electrodes providing a return electrode for the one or more energy pulses, advancing the bolus of the conducting medium to a region surrounding the feeder electrode and extending towards the target tissue site, and advancing a second bolus of the non-conducting medium behind the bolus of the conducting medium, wherein the one or more energy pulses are applied while the bolus of the conducting medium is in the region surrounding the feeder electrode.

It will be appreciated that additional advantages and modifications will readily occur to those skilled in the art. Therefore, the disclosures presented herein and broader aspects thereof are not limited to the specific details and representative embodiments shown and described herein. Accordingly, many modifications, equivalents, and improvements may be included without departing from the spirit or scope of the general inventive concept as defined by the appended claims and their equivalents.

What is claimed is:

1. A medical device, comprising:
an elongate member;
one or more expandable elements positioned on the elongate member;
one or more ports;
one or more electrodes; and
a controller, the controller being configured:
to actuate the one or more expandable elements within a vessel of a subject to block fluid flow through the vessel downstream to a target tissue site;
to deliver a bolus of conducting medium via the one or more ports towards the target tissue site; and
to apply energy pulses via the one or more electrodes for therapeutic electroporation while the bolus of the conducting medium is in contact with the one or more electrodes and extends from the one or more electrodes towards the target tissue site.

2. The medical device of claim 1, wherein the elongate member comprises a delivery catheter.

3. The medical device of claim 2, wherein the one or more expandable elements comprise one or more balloons positioned on the delivery catheter.

4. The medical device of claim 1, further comprising one or more extendable elements, the controller being further configured to advance the one or more extendable elements past a tip of the elongate member from a first site in the vessel to a second site close to the target tissue site than the first site.

5. The medical device of claim 4, wherein the one or more extendable elements comprise a guidewire.

6. The medical device of claim 4, wherein at least one of the one or more ports is positioned on at least one of the one or more extendable elements.

7. The medical device of claim 6, wherein said at least one of the one or more ports is positioned proximate a tip of said at least one of the one or more extendable elements.

8. The medical device of claim 4, wherein at least one of the one or more electrodes is positioned on at least one of the one or more extendable elements.

9. The medical device of claim 8, wherein said at least one of the one or more electrodes is positioned proximate a tip of said at least one of the one or more extendable elements.

10. The medical device of claim 4 wherein at least one of the one or more electrodes and at least one of the one or more ports are positioned on at least one of the one or more extendable elements.

11. The medical device of claim 10, wherein said at least one of the one or more electrodes and said at least one of the one or more ports are positioned proximate a tip of said at least one of the one or more extendable elements.

12. The medical device of claim 1, further comprising one or more fluid reservoirs containing the conducting medium.

13. The medical device of claim 1, wherein the controller is further configured to deliver a bolus of non-conducting medium prior to delivery of the bolus of conducting medium, the bolus of non-conducting medium flushing one or more bodily fluids from the target tissue site.

14. The medical device of claim 13, further comprising two or more fluid reservoirs, a first one of the two or more fluid reservoirs containing the conducting medium and a second one of the two or more fluid reservoirs containing the non-conducting medium.

15. The medical device of claim 1, further comprising one or more infusion pumps, wherein the controller utilizes the one or more infusion pumps to deliver the bolus of the conducting media via the one or more ports towards the target tissue site.

16. The medical device of claim 1, further comprising one or more generators, wherein the controller utilizes the generators to apply the energy pulses via the one or more electrodes for the therapeutic electroporation while the bolus of the conducting medium extends from the one or more electrodes towards the target tissue site.

17. The medical device of claim 1, wherein the one or more electrodes comprise one or more feeder electrodes and one or more return electrodes, the energy pulses being applied between respective pairs the one or more feeder electrodes and the one or more return electrodes.

18. The medical device of claim 17, wherein the one or more return electrodes are positioned on the elongate member remote from the one or more feeder electrodes.

19. The medical device of claim 17, wherein the one or more return electrodes are positioned on an additional elongate member positioned within another vessel.

20. The medical device of claim 17, wherein the controller is configured to measure impedance between the one or more feeder electrodes and the one or more return electrodes to determine when the bolus of conducting medium is in contact with the one or more feeder electrodes.

21. A system, comprising:
a first medical device comprising an elongate member, one or more expandable elements positioned on the elongate member, and one or more ports;
a second medical device comprising one or more needle electrodes; and
at least one controller, the at least one controller being configured:
to actuate the one or more expandable elements of the first medical device within a vessel of a subject to block fluid flow through the vessel downstream to a target tissue site;
to advance the one or more needle electrodes of the second medical device into the target tissue site;
to deliver a bolus of non-conducting medium via the one or more ports of the first medical device towards one or more needle electrodes of the second medical device inserted into the target tissue site to displace one or more biological fluids in a vicinity of the target tissue site; and
to apply energy pulses via the one or more needle electrodes of the second medical device for therapeutic electroporation.

22. The system of claim 21, wherein at least one of the one or more needle electrodes comprises an insulated region along a length thereof and exposed electrode region at a tip thereof.

23. The system of claim 21, wherein the at least one controller comprises:
a first controller comprised within the first medical device, the first controller being configured to actuate the one or more expandable elements of the first medical device and to deliver the bolus of non-conducting medium; and
a second controller comprising within the second medical device, the second controller being configured to advance the one or more needle electrodes and to apply the energy pulses.

24. The system of claim 21, wherein the one or more needle electrodes comprise at least a first needle electrode and a second needle electrode, and wherein the at least one controller is further configured to measure an impedance between the first and second needle electrodes to determine when the bolus of the non-conducting medium is in contact with the one or more needle electrodes.

25. A method, comprising:
delivering an elongate member of a medical device to a first site within a vessel proximate a target tissue site to be treated;
actuating one or more expandable elements of the medical device to block flow of one or more bodily fluids flow through the vessel downstream from the first site to the target tissue site;
advancing, via one or more ports of the medical device, a bolus of conducting medium, the bolus of conducting medium extending from one or more electrodes of the medical device, positioned at a second site, towards the target tissue site; and
applying, via one or more electrodes of the medical device, energy pulses to the target tissue site, the bolus of conducting medium extending a field gradient of the energy pulses from the second site to the target tissue site.

26. The method of claim 25, wherein the second site comprises a tip of one or more extendable elements of the medical device.

27. The method of claim 25, further comprising, prior to advancing the bolus of the conducting medium, advancing a non-conducting medium via the one or more ports of the medical device through the target tissue site to flush the one or more bodily fluids from the target tissue site.

28. The method of claim 25, wherein advancing the bolus of the conducting medium comprises introducing the bolus of the conducting medium via the one or more ports of the medical device and introducing additional non-conducting medium via the one or more ports of the medical device to advance the bolus of the conducting medium from the second site towards the target tissue site.

29. The method of claim 25, wherein advancing the bolus of the conducting medium comprises:
advancing a first bolus of the conducting medium through the target tissue site to extend from at least a first one of the one or more electrodes providing a feeder electrode for the one or more energy pulses to at least a second one of the one or more electrodes providing a return electrode for the one or more energy pulses;
advancing a bolus of non-conducting medium past the feeder electrode; and
advancing a second bolus of the conducting medium to a region surrounding the feeder electrode;
wherein the one or more energy pulses are applied while the first bolus of the conducting medium is in a region surrounding the return electrode and while the second bolus of the conducting medium is in the region surrounding the feeder electrode.

30. The method of claim 25, wherein advancing the bolus of the conducting medium comprises:
advancing a first bolus of non-conducting medium through the target tissue site to extend from at least a first one of the one or more electrodes providing a feeder electrode for the one or more energy pulses to at least a second one of the one or more electrodes providing a return electrode for the one or more energy pulses;
advancing the bolus of the conducting medium to a region surrounding the feeder electrode and extending towards the target tissue site; and
advancing a second bolus of the non-conducting medium behind the bolus of the conducting medium;
wherein the one or more energy pulses are applied while the bolus of the conducting medium is in the region surrounding the feeder electrode.
